SEQUENCE LISTING (22 sequences)

SEQ ID NO: 1 - DNA, 33 bp, Artificial Sequence
  Description of Artificial Sequence Primer VHpromF
atactagtcggccgcaggcacatccacagtcac

SEQ ID NO: 2 - DNA, 32 bp, Artificial Sequence
  Description of Artificial Sequence Primer VHpromR
tcccgggtatcgatggagctctcaggggattc

SEQ ID NO: 3 - DNA, 27 bp, Artificial Sequence
  Description of Artificial Sequence Primer CAintV
catcgatccgctactactactacatgg

SEQ ID NO: 4 - DNA, 18 bp, Artificial Sequence
  Description of Artificial Sequence Primer CAintR
cggccacgctgctcgtat

SEQ ID NO: 5 - DNA, 18 bp, Artificial Sequence
  Description of Artificial Sequence Primer CAMitteR
agctcacctggtgcaact

SEQ ID NO: 6 - DNA, 19 bp, Artificial Sequence
  Description of Artificial Sequence primer CAMitteF
gacctaagctgacctagac

SEQ ID NO: 7 - DNA, 18 bp, Artificial Sequence
  Description of Artificial Sequence Primer V5
tccctccaaaagctgtag

SEQ ID NO: 8 - DNA, 18 bp, Artificial Sequence
  Description of Artificial Sequence primer V6
atggcggtaatgttggac

SEQ ID NO: 9 - DNA, 18 bp, Artificial Sequence
  Description of Artificial Sequence primer V7
cacaagaatccgcacagg

SEQ ID NO: 10 - DNA, 18 bp, Artificial Sequence
  Description of Artificial Sequence Primer EBVtestR
cctgatattgcaggtagg

SEQ ID NO: 11 - DNA, 18 bp, Artificial Sequence
  Description of Artificial Sequence Primer EBVtestF
taccgacgaaggaacttg

SEQ ID NO: 12 - DNA, 377 bp, Artificial Sequence
  Description of Artificial Sequence synthetic construct
cagctggtgcagtctggggctgaggtgaagaagcctggggcctcagtgaaggtctcctgcaaggcttctggatacaccttcaccggctcctatatgcactgggtgcgacaggcccctggacaaggccttgagtggatgggacggatcaatcctaacagtggtggcacaaactatgcacagaaatttcagggcagggtcaccatgaccagggacacgtccatcagcacagcctacatggagctgagcaggctgagatctgacgacacggccgtgtattactgtgcgagagacaagctttcccggtcagaagtaccagctggccgctactactactacatggacgtctggggcaaagggaccacggtcaccgtctcctc

SEQ ID NO: 13 - DNA, 18 bp, Artificial Sequence
  Description of Artificial Sequence Primer V81
agcttcggctcaacacag

SEQ ID NO: 14 - DNA, 18 bp, Artificial Sequence
  Description of Artificial Sequence Primer V83
gccttacctgcagagatg

SEQ ID NO: 15 - DNA, 22 bp, Artificial Sequence
  Description of Artificial Sequence Primer V89
agtataccccagaactctgctt

SEQ ID NO: 16 - DNA, 30 bp, Artificial Sequence
  Description of Artificial Sequence Primer V90
ggccgctgcggccggaagatgaggctgact

SEQ ID NO: 17 - DNA, 25 bp, Artificial Sequence
  Description of Artificial Sequence Primer V91
agcggccgcttgcaggacaatatga

SEQ ID NO: 18 - DNA, 18 bp, Artificial Sequence
  Description of Artificial Sequence Primer V94
ttgcgtgacaggctcagt

SEQ ID NO: 19 - DNA, 18 bp, Artificial Sequence
  Description of Artificial Sequence Primer V115
atcacacggcacttctcg

SEQ ID NO: 20 - DNA, 25 bp, Artificial Sequence
  Description of Artificial Sequence Primer V116
gagatatcggcttctggaggacact

SEQ ID NO: 21 - DNA, 14000 bp, Artificial Sequence
  Description of Artificial Sequence Proposed sequence of the arranged light chain locus
ctctccagcaaggggataagagaggcctgggaggaacctgctcagtctgggcctaaggaagcagcactggtggtgcctcagccatggcctggaccgttctcctcctcggcctcctctctcactgcacaggtgatccccccagggtctcaccaacctgcccagcccaagggttctgggtccagcgtgtccttgattctgagctcaggagggcccttcctgtggtgggcaggatgctcatgaccctgctgcagggtgggaggctggtggggctgaactccccccaaactgtgctcaaaggcttgtgagagcctgagggactgcacctgccaggagagagtagtgagttttcagttcaaagtctccatacaacaggaaagtcatgggccactggggctggggctgattgcaggggataccctgagggttcacagactctctggagcttgtctgggacagcagggcaagggatttcataagaagcatctttcacctgcaagccaacctctctcttatttatttatttatttatttatttatttatttatttatttttatctttgcaggctctgtgacctcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatcccacggtgacacaggcagatgaggaagtgagacaaaaacaccctcccagcctcggtcaccctcttgctccagccccgggaagcctgttgataaagccatgagtgaatctggcccagttcacctggatctgagcctttcaggttgcccttccctccagccccctccaggagtctctacagaagatacatcaggcataaatatggcctggaagggccagaatcatctggtgacttggggctgttgtgtgagttagagaatgaaggcttgggtggaaagacagacagaggcaacctctgtccactgtcctacccctggatggtcatatggtggggacagggcaagtccttagaccaactgtctggatcaggccccagaactactgcccagttctgctgaggtcctggcccccaggctgtgtggcagcctgtgattcccaacagagcaaaccagaggaatggacactgtgaagtctgcccagatcccctcctcaatgtgacccacctggcactgctgagaagcccagcagctcagagctgtgccctcactgggaagtgctgttggttgcagaaagcttcctcaagtttgtgtcccttttcagaggggttcggtttaatcaaccaagatctcaaatccttgcctcaatttaagatgccactgaatgaagggcctcccagctccagagctccctgtgtggatacctgaggcctcaatgtcaactccatcacgagtcagggtctccttctgccccgtgttgcctcccccactcccttctgaatcttctgtgcatggacatctctattgcagagttagcttccagagaaccccatctaagatggccagctgtccccaacatgggtcatcagggacctgagtaggccactataaactgaaaactctggtttctgtccaaatttgcagagtaaatgttgaaatgcccaatctgatggttccttgaatttttatggaatgaaaagggagcctgacatgccaggtgctctgggttgagggattgttggagtcagatctccctgcaggaaagcccggggcagggggagcagcctcacccctcacaggaaccacagatacacccacaaggtgagctgcaggatggatgctgcccacctccaccctccacatcctctgtaaatgttgctcctttctacaactccaaccagatatgtagatgtggcgaactacgtaaaatacggatcattcatcacatcaaaacccactgcaggacaccctggtcaacaaagaacccaatcacatccccatcaactacatagtttccaaattttccatctccagaaaaataacaataacaatatacatgaaaatcgatgtaatttatctcatacataatttcatgttgataacgtgaaaatgatagtattttgctctactgaaataaataaaatatatatatatatctgaatttatttgtcttatcttttcatatttaatgtggtgactagacactagggggtcacaggaggatcgtgtcataccactatgggacagagctcctcacaactctttcaggtgacaggtactgttgagtaacctgctgcaagcatccccatctccaccagaccatataagtgtgaacccaggaagaggcactggaacaatagagagaaaaacctgcttgtgcagaagacggtgcccttgagccctgctcctgctccatcctacgggtgccacattcatctcatggtgtaatatttcgtgccctgcctgagcttatgaccgaggggatatggcaggtctgactgtgtggttactggtgtctcatgaggttctggatgtaacaaagccctcgaatatagaagaggttgttttcaaaaggaaataattatctactgcacatgacatagacttgttgctaaatcccatgcgtctacactaggattctcctctgaagccttgccttaagcacaaggtttcagttcctatgtccagttcctctattatggtagagtctgctagtttctctggcccaatagcaggacactcactcccccacctgcacctgctgcagagcctttctactcttggccccaaaacactgggtgacacagttctcagacccatgatttatagtgtcagtattcaggcctcaggggtccctgatggcttctctggctccaagtctggaaacacagcctccatgaccatctctgggttccaggctgaggatgaggctgattattactgcaactcacataggagaggtggcactttccaccgtggtccaagttcatggggaattgagacccaaacctgccctgggctctcagcctctctcttgttctgaagatgcttcctcaccctgtgcaaggggcttcttgcagcactgccttgagaatttcccctctcccagctcctctcctttctcaccaggaagtccaaaaggaaacctgctctgtgatttctcatccaggacagtgacagcttcctgatgcttgtgtgctgtggtccctgaatgtgcaactcttcctagctcttcaaatgcaggcacatagtgagaaaagctgcctgactggtgcattcactgctgtttttaaggatgtcctcacccaaaatgcatcctcctcccaaattgtgaagaacaatctggacagaggtcattacagggagtttcaagaaactgcatcttattcaattgtgtccaccatggtctggtaaagatggccctcctggatggactattcctctgcatgtctgtcctgaagcagtgaccactgtgagaagatctgaacatgtttgtgaggtattaaggacgagaggaaactgttgtttttattattcttttgtttttgtttttgaaacaaacttttgctttgtcgccaggctggagtgcagtggcagaatcttggcttactgaaatctcagcctcccaggctctaccaatgctccctgcctcagcctcccgaagagctgggataacaggtgaccaccaccatgcctggctgatttttgtatatttagtagagacgggatttcaccatgttggccaggctggtcttgaactcctgatctcaggtgatgcacccacctcggcctctcaaagggatgggaatacacacaggagccactgcatctggcagtgttttttttatttttgctcctcctctttgcctcaatacctcaggttgctgagctggggagattttgcgtgacaggctcagtgctccctcaaaatcctcccgtctcaattcgctggggccctgtcctggaaactccccaaaagtggatggtgtccctataggttgggagtttccaaaatggccccacagggaagagttaacgtgagtccattccttcttcctcattgacatccagcatttgtaatttccatgggtgtcaatacttttgtagctgaaatctttcttaatctactaaaggtgagaatgaatttaataaatattcagacattagttgcatccaatatttaaattttatgagtcaattggtagacatagccattattatatataatttaggcttcataaactttgattaaataggttttattaaaaacaaagtaaccattttattatgtgtttagactatatcaacatgttgtgtacctgaaatatccacaagaaaatatatttcaaaaaccaaattgtatttattgtctattgttgcataaaaattgctccctaatatttagcatggtaagagaacacgtgtttgtgatcttgtcacttcggtgcatctggaattgagagcagcttagttttgtggttctggttctgggttggtcatgaagttgcagccaagctgtcaggccaggctgcattcagaggccagagcaggtggccaggcccagcctgaggggcttccactgtccctaacctgtttgtctgatgtggaaaatctcagaggaaaaggagagagtgaagtgtaaggcacctgtcccagtcccccttgtcaaaggccatcccatacctgcaccatttcttattctttcctggggcgtcataggcatagagcactgcccattcattctaacgctgtagagtattctgtagtaggattttagccatgcagcctctaatggttatcaccatgattttgatcttacaaatcacactgcagcaagcatccctgtgcagactcctttgagttcatgtgtgcatatcaccataggataaatttccagaagtggaattgctgggtcaaaaggatgtgcatttttaacttttatccattgttttcatattcccctccagttctaccagtttacaataccagccctaaatattgattggaattcattggtgaaagtgcaagtttgtgccaacctatcagatatacaaagttatcttgttacacatttatttttgatttctcctattttgcttgaggttgagcatttactcaaatatttcagtgctcattatgttttaggatttggtaaaccgtttcttcaatgcctcggtaagaggtattttagtctttgccatgcacaagacaacgttgggataatatgtatgttctgatacaccatctacaacgtaatcattaaaacatataaaaaccactatcagttctggggccattaaaaaaattggtggcaggccagggatgtcccacaggatgtggtttaacggtctctggtttctagggttatttgaagtttgaacattgcacccgcatatgttctatgtggagatctctttgtgagggacactgtaattcacctcctctaggggcctgaggtctttctttggataagaacctacctgtaccatgtgtttgattggatcttgtgtctgctcaagacagccctgtgtcacaagctcatgactttcatcttcatccatttgctctgttttgtgagagcttcagtatatcaggaatagagattcctccgaggtgaaaaattagaggcagagggaggggcaaatgggcaaggaagcttgcaccaagtcgggagtgatccagtgtaggctgagagaaaaaaggtcttaaaatcagccttgtagctgaaaccaaaaacacacaagatggttggtgttctgagcatcattaacaaatgataaatgaagttgaacttttaaatgtattgcaaatttttataaagcaagtagatcgttaaactcagaatgcaacaatggaataaagaagagagtttgagatgtttttaaaatttatttatttatttatttatttttgagatggagtctcactctgttgccaggctggagtgcagtggcacaatcgcggctcactgcaacatccacctcccgggttcaagaaattctcctgcctcagcctcccaagtagctgggattacaagctctcgccatcatgccaggctaatttttgtattttttgtagagacatggtttcacaatgttgaccaggatggtgtcaatctcctgacctcatgatccacccgtctcggcctcccaaagtgctgggattacaggcatgaactaccgtgcctggctgagtttgagattttaactgtaagtcctccaactaagttgccatgacaagaacagggatgatgagagtggaaatatgttatcctgcaaattatcgttttatgtaaaagaatattttccctcttttaggtaaaggaagcatcttctggagcaccttctctctgactatcaaagcaccattaagccacaaataaactgtaacatgaagtaggaaacaactgcccttttatataaccattgagaggtggctttatatgcataccaaaatgttgatgctcaatgctaaaattggatttagtaatttaatatgcctacaagaaattaattttctttggattatattatttctgtgtacgatttatcttagttaacttggaaatattctgctctaaaaacaactcttgttttttgggttatattttctgtatcaactatagctcttttccaaatgctgtcagagatagcccatggctactgatcacaaaattcaattttatggcatttaaattattctatactctaaattattttaaaagtgcacagatgtgaatttttcacatctgactcaaaaatgttgctgatgttgactcacttttttatttcaatcttattgaagtaggagtttacttttctggaacctggatgataacaggagactggagaggaaaccccccaaattgttttcctttaaaccctcaggatgaatcatcctggataatcacccacacttgatttgggtgatatctaaatgagagttgggtcttagagtaggtgctgagttagtttaggacttgcgctgttggaatgagttgaatgtttttacaagtgagaaagacatgagttttttggagtccagagggtggggggttattggctgaattaagtcccccaaaatgtatgcattgaagctgtaacacacaatatgtgactgaaattgtgcatagggtctttaaagaggtgactaagtgaaaatgaaaaaattagggtggattcttctcaaattggactgatgtcctcctaggaagaagaaatttgcacacacagaaatgaggcaccagaggtgagcgtgcagagaaaagaccaggtgaggattcagcaaggaggtagcaacctgcaagccaaggagagagtcctcaggggaaaccaaacccactaccacctttatcttgggttttccagcttcagaactgtgagaaaatatgtttctgccatttcggtcactaattctttcctatcttcttgtgggagctctagcaaaaacaagagggaccccaaagaccttggatgagggagaaggaggagatggagcagggtgcaggaggcggtgcaggaaggggctggaaggtcgggctctgaggtgcatctcctgggtggaatcttgactccactccctattgtctggaggacttgggaaaaacatttaacctcctaatattcactcactaataaagatgggcttgaagcacaaggctccccatcatcctattctatattacaaaagtcttcttgaggtaacacttgtaaaactctcgctaatgcatctggcatgtattatggactcataagtagcccttctgagtgatctagtgatgtgcagaaaatggcattcatgctgtgtgcaccagggggcactgtgaggtttagtctgaggcccctaatgagtccaagcccctagtaatgctcaagggcgaagagcctgactgttgcttcctatgaggccccttctagtgggtaaatctgaaaatgcacttggcccttcttctgatcttgagaaattactcagagaaggccatcaggctcagggctcagacaagaaccaggacaaatgttttagggaatggagaacagatttgcatccactgctcaccagagccacctaacgacgacacaagaataaaggaagtagatttgcatgaagagacttcccttcctatgataagagaggcctggaggttcctccttagctgtgggctcagaagcagagttctggggtgtctccaccatggcctggacccctctctggctcactctcctcactctttgcataggtgctgcctcccagggctcaaccccatattatcatgctagctgtgccaacctggccctgagcttcggctcaacacagggagtagtgtagggtgtgggactctaggcgtgaaacccttatcctcacctcttctgtcctcttttgcataggttctgtggtttcttctgagctgactcaggaccctgctgtgtctgtggccttgggacagacagtcaggatcacatgccaaggagacagcctcagaagctattatgcaagctggtaccagcagaagccagggcaggcccctatacttgtcatctatgataaaaacaaccggccctcagggatcccagaccgattttttggctccagctcaggaaacacagcttccttgaccatcactggggctcaggcggaagatgaggctgactattactgtaactcccgggacagcagtggtcaccgtgtggttttcggcggagggaccaagctgaccgtcctaggtgagtctcttctcccctctccttccccgctcttgggacaatttctgctgtttttgtttgtttctgtatcttgtctcaacttgtggtcagcctttctccctgcatcccaggcctgagcaaggacctctgccctccctgttcagacccttgcttgcctcagcaggtcactacaaccacttcacctctgaccacaggggcaggggactagatagaatgacctactgagcctcgtctgtctgtctgtctgtctgtctctctgtttgtctctctgtctctctgtttgtctctctgactgtctgacaggcgcaggctgggtctctaagccttgttctgttctggcctcctcagtctgggttcttgtcggaacagctttgtccttgggttacctgggttccatctcctggggaattgggaacaaggggtctgagggaggcacctcctgggagactttagaaggacccagtgccctcggggctgatgctcgggaatcacagagctgggacccagagccaggatccagacccagaatgaggtaggaggtggaggggctgccctgggcgtctgggggctgccagggactgagccctgagccagcctgagactcaggaaaccccgtcaggagggagaagggagaagcagactctggacaccagaaagccaggggaagggtcacaaaaggagtggatgtgacggaagggcgggctcctgggtctcttcagaacatatcccctgtgcccagggggatcagaggggcagagtccactgcgtgaaagccccactgctatgaccaggtagccgggacgtggggtggatgccagaaaagactccacggaataagagagagcccaggacagcaggcaggctctccgatccccccaggcccttgccccatacacgggctccagaacacacatttggctggaacagcctgagggaccaaaaggccccagtatcccacagagctgaggagccaggccagaaaagtaaccccagagttcgctgtgcaggggagacacagagctctctttatctgtcaggatggcaggaggggacagggtcagggcgctgagggtcagatgtcggtgttgggggccaaggccccgagagatctcaggacaggtggtcaggtgtctaaggtaaaacagctccccgtgcagatcagggcatagtggaaaacaccctgacccctctgcctggcatagaccttcagacacagagcccctgaacaagggcaccccaacacctcatcatatactgaggtcaggggctccccaggtggacaccaggactctgaccccctgcccctcatccaccccgcaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggttctcaaccctcaccccccaccacgggagactagagctgcaggatcccaggggaggggtctctcctcccaccccaaggcatcaagcccttctccctgcactcaataaaccctcaataaatattctcattgtcaatcagaaatcttgttttatctcattttttcttttctcacatataattcctagcctttcctgggttctcaatttgtggtggaaagaaccctgaacccagtgggaaagttgcctatgtgaaggggttctcagttccctgggcatctctgcaggtaaggccttcctcacccagacaccccttcctcagctctccactgtacccctgagccaccagcctcgcctggctgggaccaggggggtgtcacactctcctagattctgcctttcaacagaaacctaaccacgcatcacacggcacttctcgcatgccttctgtgtctgctccagtctctgggctaaagagttgctggtccgggacaggggataggtccgctcttggtcagatgccaggtccctgccatggcatccctgaccctatgcaacaagccagtgactctggtgagctctctgtgtcaggagaatccatgatccagagtttcatattgtcctgcaagcatctggtgggctgtagctcttgccaaactgggaaataccatggcccagcatcaggatgcaggacagtccggagagggaaatcaggagaagtgaaggggtctctggggagcccagatgtgggctagaggcagaagtaagggtgaagagcacctatgagtcaatgtcatggtctcagcaggaacacagttgaaaatccccattccacacaagaccgtttagcaggaaaggagtccatacttgtgctgccaccaggatgtcctgagaagccttggagaatgaaacatacaggtgcatttcctagacttgacaatgcacgttagccaagtaaaggcaatgaaaagttctctactagggaaataatttcctgtggtaaagcttagcttatgtaaagtcacatttatccatctggcacctctaaaagccccataatattctgcaagatactagtatgtcatggaagtagtttatgaaacataaagtgagatttaagaacaaagatgttacgggtgtatgataagatggctacaggctcagggtcaggctcgaggagtgaaggaggccgtgtcaaattcatgacaagagttggagctgggccaggctgggtcagggctgtgtgaatgcagacagagggctacaggcaaggtcaggcatccatgaacactcagctcccccagaccctcctgcccactgggaccttcgccctcccttggtcacagtggtggagccttcctacccaaacctctatggaggccctggatgactgtgcgttcttagtgcccacgcaaacttagactccctgtctctgcctccagcacatcaggaatgtggcagctgagttcaccagagctgctgggtggtcccgacaggccagggacagagcccgcaaagacaggaagctctgcagtcacaatgaggcagagaaatggccccttggtgcttgatcacagccacccctgatccaaatcccagcctctgaattagaagaaggctaaaaggttctagtggccacagtccctgtctaagcccatttcacaaatgagaaaactaagaccacccaaggagggccagttacgtaggcctgctgggtacaaggccaaggtctacttcacacccagcagctgtccaaagactgagctgtgtcataagtttatattatgaagaactctgaacatataaataaggagacagaaaaataacagtgtcccatgttctcatcacccagcactcaaaataagcaattcacagatgatgccgacccacccacagcaaaataaattctcccttacacaacatttagaaagaaatacaagacatcagatctgttcagctgtaagtactccattactgtcctggaatgacatggaccttaaaataactataatatcactaccaaacctaaatagaaattatcactaattccctaatatcgagaaataagcagggtctcctcaaatgcatcagaaacaccagaagtgctttggcttagttacatgttggtgctgttggtatttgggggtttaagtttatatgaggagcaatatgacatcaaatggtgatgggtgcatgtgccatcaggctggttgtcactggtgaatatttcctcaattgctctagagcctcccggcaaggcaggagctgcaggagctgagagctgtctggagaacttcccctggctgctatacagccacgcctcctggagcaggaacctagggcttccctcagcttttattttcctggaaaatgattctagcatgaaggggattaacttgattcagattggacattgcaaaatagcttgcaaggacagggagctgctaccagcagagtcacccatgtcagactgccactcttgtagtaatgttagctgcataggatggtcaatagctacatccctcagaagggaaggaaggcagagggttgaggcttcagttcacctccttctcatgagtgctgcagagtgtctgtgatgtcagaggtctgcagctgggctctgttcacccaggagtgtgcttcatgctctaggaaggagccactttgcacacagaagatccggggcccagccatccttccagggtgaacaattcatgtcttctctcatggtgaactctaggattcaagccatctaatgcttttgaagccactgtcattatatttaattgatgatgacaggtggccaccaatgatgaatattttcccagggggagtctccccaagtggcttcagacttcctcacatggccccaggggattaaatggctcctgattactcagaggataagaggttctgtcttatcatgttcctttcttatttgtcttatgtgtctttcctgccccaggcctgggatcccccactgatctcccttcccttagtgagaggtgatatttggagaccacattctggaggctccctcatgtcccccatttgaaaaagacaacggcagcctccaccctagctgtcccacccaacatgaggccagattcaggggtgcagggatgctcccaaggttaccctaacagatgtgactggcacttcatattgggaccagccaggcctcactgaccaggcctatccaactagaactactccagaaggtggggctgaaacccaccaaggttcccagaacactgcactctagggcaatcagcctctgcatgggaggagaggagcaccctctgcaccaccccatggtgttaccaaaagttgaaccatgggttggttcaactttgcagagaagagaccacctatcccatctgtggaaattcactccttagcgacactaatgccctctaataaattcaatcctgggcctgagtgatggttggtgcaaaaaacaaattcaagatcccagtgtcctccagaagcctggatttccaggg

SEQ ID NO: 22 - DNA, 13685 bp, Artificial Sequence
  Description of Artificial Sequence Vector pVLCL
ctagtcctgcaggtttatcggcttctggaggacactgggatcttgaatttgttttttgcaccaaccatcactcaggcccaggattgaatttattagagggcattagtgtcgctaaggagtgaatttccacagatgggataggtggtctcttctctgcaaagttgaaccaacccatggttcaacttttggtaacaccatggggtggtgcagagggtgctcctctcctcccatgcagaggctgattgccctagagtgcagtgttctgggaaccttggtgggtttcagccccaccttctggagtagttctagttggataggcctggtcagtgaggcctggctggtcccaatatgaagtgccagtcacatctgttagggtaaccttgggagcatccctgcacccctgaatctggcctcatgttgggtgggacagctagggtggaggctgccgttgtctttttcaaatgggggacatgagggagcctccagaatgtggtctccaaatatcacctctcactaagggaagggagatcagtgggggatcccaggcctggggcaggaaagacacataagacaaataagaaaggaacatgataagacagaacctcttatcctctgagtaatcaggagccatttaatcccctggggccatgtgaggaagtctgaagccacttggggagactccccctgggaaaatattcatcattggtggccacctgtcatcatcaattaaatataatgacagtggcttcaaaagcattagatggcttgaatcctagagttcaccatgagagaagacatgaattgttcaccctggaaggatggctgggccccggatcttctgtgtgcaaagtggctccttcctagagcatgaagcacactcctgggtgaacagagcccagctgcagacctctgacatcacagacactctgcagcactcatgagaaggaggtgaactgaagcctcaaccctctgccttccttcccttctgagggatgtagctattgaccatcctatgcagctaacattactacaagagtggcagtctgacatgggtgactctgctggtagcagctccctgtccttgcaagctattttgcaatgtccaatctgaatcaagttaatccccttcatgctagaatcattttccaggaaaataaaagctgagggaagccctaggttcctgctccaggaggcgtggctgtatagcagccaggggaagttctccagacagctctcagctcctgcagctcctgccttgccgggaggctctagagcaattgaggaaatattcaccagtgacaaccagcctgatggcacatgcacccatcaccatttgatgtcatattgctcctcatataaacttaaacccccaaataccaacagcaccaacatgtaactaagccaaagcacttctggtgtttctgatgcatttgaggagaccctgcttatttctcgatattagggaattagtgataatttctatttaggtttggtagtgatattatagttattttaaggtccatgtcattccaggacagtaatggagtacttacagctgaacagatctgatgtcttgtatttctttctaaatgttgtgtaagggagaatttattttgctgtgggtgggtcggcatcatctgtgaattgcttattttgagtgctgggtgatgagaacatgggacactgttatttttctgtctccttatttatatgttcagagttcttcataatataaacttatgacacagctcagtctttggacagctgctgggtgtgaagtagaccttggccttgtacccagcaggcctacgtaactggccctccttgggtggtcttagttttctcatttgtgaaatgggcttagacagggactgtggccactagaaccttttagccttcttctaattcagaggctgggatttggatcaggggtggctgtgatcaagcaccaaggggccatttctctgcctcattgtgactgcagagcttcctgtctttgcgggctctgtccctggcctgtcgggaccacccagcagctctggtgaactcagctgccacattcctgatgtgctggaggcagagacagggagtctaagtttgcgtgggcactaagaacgcacagtcatccagggcctccatagaggtttgggtaggaaggctccaccactgtgaccaagggagggcgaaggtcccagtgggcaggagggtctgggggagctgagtgttcatggatgcctgaccttgcctgtagccctctgtctgcattcacacagccctgacccagcctggcccagctccaactcttgtcatgaatttgacacggcctccttcactcctcgagcctgaccctgagcctgtagccatcttatcatacacccgtaacatctttgttcttaaatctcactttatgtttcataaactacttccatgacatactagtatcttgcagaatattatggggcttttagaggtgccagatggataaatgtgactttacataagctaagctttaccacaggaaattatttccctagtagagaacttttcattgcctttacttggctaacgtgcattgtcaagtctaggaaatgcacctgtatgtttcattctccaaggcttctcaggacatcctggtggcagcacaagtatggactcctttcctgctaaacggtcttgtgtggaatggggattttcaactgtgttcctgctgagaccatgacattgactcataggtgctcttcacccttacttctgcctctagcccacatctgggctccccagagaccccttcacttctcctgatttccctctccggactgtcctgcatcctgatgctgggccatggtatttcccagtttggcaagagctacagcccaccagatgcttgcaggacaatatgaaactctggatcatggattctcctgacacagagagctcaccagagtcactggcttgttgcatagggtcagggatgccatggcagggacctggcatctgaccaagagcggacctatcccctgtcccggaccagcaactctttagcccagagactggagcagacacagaaggcatgcgagaagtgccgtgtgatgcgtggttaggtttctgttgaaaggcagaatctaggagagtgtgacacccccctggtcccagccaggcgaggctggtggctcaggggtacagtggagagctgaggaaggggtgtctgggtgaggaaggccttacctgcagagatgcccagggaactgagaaccccttcacataggcaactttcccactgggttcagggttctttccaccacaaattgagaacccaggaaaggctaggaattatatgtgagaaaagaaaaaatgagataaaacaagatttctgattgacaatgagaatatttattgagggtttattgagtgcagggagaagggcttgatgccttggggtgggaggagagacccctcccctgggatcctgcagctctagtctcccgtggtggggggtgagggttgagaacctatgaacattctgtaggggccactgtcttctccacggtgctcccttcatgcgtgacctggcagctgtagcttctgtgggacttccactgctcaggcgtcaggctcagatagctgctggccgcgtacttgttgttgctttgtttggagggtgtggtggtctccactcccgccttgacggggctgctatctgccttccaggccactgtcacggctcccgggtagaagtcacttatgagacacaccagtgtggccttgttggcttgaagctcctcagaggagggcgggaacagagtgaccgagggggcagccttgggctgacctgcggggtggatgaggggcagggggtcagagtcctggtgtccacctggggagcccctgacctcagtatatgatgaggtgttggggtgcccttgttcaggggctctgtgtctgaaggtctatgccaggcagaggggtcagggtgttttccactatgccctgatctgcacggggagctgttttaccttagacacctgaccacctgtcctgagatctctcggggccttggcccccaacaccgacatctgaccctcagcgccctgaccctgtcccctcctgccatcctgacagataaagagagctctgtgtctcccctgcacagcgaactctggggttacttttctggcctggctcctcagctctgtgggatactggggccttttggtccctcaggctgttccagccaaatgtgtgttctggagcccgtgtatggggcaagggcctggggggatcggagagcctgcctgctgtcctgggctctctcttattccgtggagtcttttctggcatccaccccacgtcccggctacctggtcatagcagtggggctttcacgcagtggactctgcccctctgatccccctgggcacaggggatatgttctgaagagacccaggagcccgcccttccgtcacatccactccttttgtgacccttcccctggctttctggtgtccagagtctgcttctcccttctccctcctgacggggtttcctgagtctcaggctggctcagggctcagtccctggcagcccccagacgcccagggcagcccctccacctcctacctcattctgggtctggatcctggctctgggtcccagctctgtgattcccgagcatcagccccgagggcactgggtccttctaaagtctcccaggaggtgcctccctcagaccccttgttcccaattccccaggagatggaacccaggtaacccaaggacaaagctgttccgacaagaacccagactgaggaggccagaacagaacaaggcttagagacccagcctgcgcctgtcagacagtcagagagacaaacagagagacagagagacaaacagagagacagacagacagacagacagacgaggctcagtaggtcattctatctagtcccctgcccctgtggtcagaggtgaagtggttgtagtgacctgctgaggcaagcaagggtctgaacagggagggcagaggtccttgctcaggcctgggatgcagggagaaaggctgaccacaagttgagacaagatacagaaacaaacaaaaacagcagaaattgtcccaagagcggggaaggagaggggagaagagactcacctaggacggtcagcttggtccctccgccgaaaaccacacggtgaccactgctgtcccgggagttacagtaatagtcagcctcatcttccggccgcagcggccaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacaaacgaattcgcccttagtataccccagaactctgcttctgagcccacagctaaggaggaacctccaggcctctcttatcataggaagggaagtctcttcatgcaaatctacttcctttattcttgtgtcgtcgttaggtggctctggtgagcagtggatgcaaatctgttctccattccctaaaacatttgtcctggttcttgtctgagccctgagcctgatggccttctctgagtaatttctcaagatcagaagaagggccaagtgcattttcagatttacccactagaaggggcctcataggaagcaacagtcaggctcttcgcccttgagcattactaggggcttggactcattaggggcctcagactaaacctcacagtgccccctggtgcacacagcatgaatgccattttctgcacatcactagatcactcagaagggctacttatgagtccataatacatgccagatgcattagcgagagttttacaagtgttacctcaagaagacttttgtaatatagaataggatgatggggagccttgtgcttcaagcccatctttattagtgagtgaatattaggaggttaaatgtttttcccaagtcctccagacaatagggagtggagtcaagattccacccaggagatgcacctcagagcccgaccttccagccccttcctgcaccgcctcctgcaccctgctccatctcctccttctccctcatccaaggtctttggggtccctcttgtttttgctagagctcccacaagaagataggaaagaattagtgaccgaaatggcagaaacatattttctcacagttctgaagctggaaaacccaagataaaggtggtagtgggtttggtttcccctgaggactctctccttggcttgcaggttgctacctccttgctgaatcctcacctggtcttttctctgcacgctcacctctggtgcctcatttctgtgtgtgcaaatttcttcttcctaggaggacatcagtccaatttgagaagaatccaccctaattttttcattttcacttagtcacctctttaaagaccctatgcacaatttcagtcacatattgtgtgttacagcttcaatgcatacattttgggggacttaattcagccaataaccccccaccctctggactccaaaaaactcatgtctttctcacttgtaaaaacattcaactcattccaacagcgcaagtcctaaactaactcagcacctactctaagacccaactctcatttagatatcacccaaatcaagtgtgggtgattatccaggatgattcatcctgagggtttaaaggaaaacaatttggggggtttcctctccagtctcctgttatcatccaggttccagaaaagtaaactcctacttcaataagattgaaataaaaaagtgagtcaacatcagcaacatttttgagtcagatgtgaaaaattcacatctgtgcacttttaaaataatttagagtatagaataatttaaatgccataaaattgaattttgtgatcagtagccatgggctatctctgacagcatttggaaaagagctatagttgatacagaaaatataacccaaaaaacaagagttgtttttagagcagaatatttccaagttaactaagataaatcgtacacagaaataatataatccaaagaaaattaatttcttgtaggcatattaaattactaaatccaattttagcattgagcatcaacattttggtatgcatataaagccacctctcaatggttatataaaagggcagttgtttcctacttcatgttacagtttatttgtggcttaatggtgctttgatagtcagagagaaggtgctccagaagatgcttcctttacctaaaagagggaaaatattcttttacataaaacgataatttgcaggataacatatttccactctcatcatccctgttcttgtcatggcaacttagttggaggacttacagttaaaatctcaaactcagccaggcacggtagttcatgcctgtaatcccagcactttgggaggccgagacgggtggatcatgaggtcaggagattgacaccatcctggtcaacattgtgaaaccatgtctctacaaaaaatacaaaaattagcctggcatgatggcgagagcttgtaatcccagctacttgggaggctgaggcaggagaatttcttgaacccgggaggtggatgttgcagtgagccgcgattgtgccactgcactccagcctggcaacagagtgagactccatctcaaaaataaataaataaataaataaattttaaaaacatctcaaactctcttctttattccattgttgcattctgagtttaacgatctacttgctttataaaaatttgcaatacatttaaaagttcaacttcatttatcatttgttaatgatgctcagaacaccaaccatcttgtgtgtttttggtttcagctacaaggctgattttaagaccttttttctctcagcctacactggatcactcccgacttggtgcaagcttccttgcccatttgcccctccctctgcctctaatttttcacctcggaggaatctctattcctgatatactgaagctctcacaaaacagagcaaatggatgaagatgaaagtcatgagcttgtgacacagggctgtcttgagcagacacaagatccaatcaaacacatggtacaggtaggttcttatccaaagaaagacctcaggcccctagaggaggtgaattacagtgtccctcacaaagagatctccacatagaacatatgcgggtgcaatgttcaaacttcaaataaccctagaaaccagagaccgttaaaccacatcctgtgggacatccctggcctgccaccaatttttttaatggccccagaactgatagtggtttttatatgttttaatgattacgttgtagatggtgtatcagaacatacatattatcccaacgttgtcttgtgcatggcaaagactaaaatacctcttaccgaggcattgaagaaacggtttaccaaatcctaaaacataatgagcactgaaatatttgagtaaatgctcaacctcaagcaaaataggagaaatcaaaaataaatgtgtaacaagataactttgtatatctgataggttggcacaaacttgcactttcaccaatgaattccaatcaatatttagggctggtattgtaaactggtagaactggaggggaatatgaaaacaatggataaaagttaaaaatgcacatccttttgacccagcaattccacttctggaaatttatcctatggtgatatgcacacatgaactcaaaggagtctgcacagggatgcttgctgcagtgtgatttgtaagatcaaaatcatggtgataaccattagaggctgcatggctaaaatcctactacagaatactctacagcgttagaatgaatgggcagtgctctatgcctatgacgccccaggaaagaataagaaatggtgcaggtatgggatggcctttgacaagggggactgggacaggtgccttacacttcactctctccttttcctctgagattttccacatcagacaaacaggttagggacagtggaagcccctcaggctgggcctggccacctgctctggcctctgaatgcagcctggcctgacagcttggctgcaacttcatgaccaacccagaaccagaaccacaaaactaagctgctctcaattccagatgcaccgaagtgacaagatcacaaacacgtgttctcttaccatgctaaatattagggagcaatttttatgcaacaatagacaataaatacaatttggtttttgaaatatattttcttgtggatatttcaggtacacaacatgttgatatagtctaaacacataataaaatggttactttgtttttaataaaacctatttaatcaaagtttatgaagcctaaattatatataataatggctatgtctaccaattgactcataaaatttaaatattggatgcaactaatgtctgaatatttattaaattcattctcacctttagtagattaagaaagatttcagctacaaaagtattgacacccatggaaattacaaatgctggatgtcaatgaggaagaaggaatggactcacgttaactcttccctgtggggccattttggaaactcccaacctatagggacaccatccacttttggggagtttccaggacagggccccagcgaattgagacgggaggattttgagggagcactgagcctgtcacgcaaaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaaggga

(19) United States
(12) Patent Application Publication
(10) Pub. No.: US 2006/0148085 A1
(43) Pub. Date: Jul. 6, 2006

(54) High yield heterologous expression cell lines for expression of gene products with human glycosylation pattern

(76) Inventors: Volker Sandig, Berlin (DE); Karsten Winkler, Berlin (DE); Uwe Marx, Berlin (DE); Tobias Wermelinger, Hochsule Wadenswil (CH)

Correspondence Address: NEEDLE & ROSENBERG, P.C., SUITE 1000, 999 PEACHTREE STREET, ATLANTA, GA, 30309-3915, US

(21) Appl. No.: 10/530,224

(22) PCT Filed: Oct. 6, 2003
(86) PCT No.: PCT/EP03/11027

§ 371 Date: Jul. 21, 2005

(30) Foreign Application Priority Data
Oct. 4, 2002 (EP) 02022194.1

(51) Int. Cl. C12N 5/08; C12N 15/87

(52) U.S. Cl. 435/455; 435/366

(57) ABSTRACT

The invention relates to ubiquitous/universal processes for establishing cells capable of stable high yield expression of a recombinant gene with human glycosylation pattern, and for establishing stable universal precursor cells available for insertion of arbitrary target genes. The invention further relates to cells obtainable by said processes

### TECHNICAL FIELD

The present invention relates to ubiquitous/universal processes for establishing cells capable of stable high yield expression of a recombinant gene with human glycosylation pattern, and for establishing stable universal precursor cells available for insertion of arbitrary target genes. The invention further relates to the cells obtainable by said processes.

### BACKGROUND OF THE INVENTION

Recombinant protein production is of central importance for different applications. Structural studies of proteins (rational drug design and drug optimisation are based thereon (Antivir. Chem. Chemother., 12 Suppl. 1, 43-49 (2001))), industrial applications of proteins (enzymes) and clinical use of recombinant proteins have increased the need for their efficient production. As of February 2000, according to a survey by the Pharmaceutical Research and Manufacturers of America, 122 biologics, including 20 monoclonal antibodies were either in phase III trials or awaiting FDA approval (K. Garber, 2001, Nature Biotech. 19, 184-185).

Depending on the application, native conformation and correct posttranslational modifications (such as glycosylation) of the recombinant protein are essential. Prokaryots such as the biotechnology “pet” organism Escherichia coli (E. coli) lack the ability to introduce posttranslational modification. Only eukaryotic cells possess the cell machinery necessary for co-translational and post-translational modifications as they are often required to produce functionally active proteins. Various eukaryotic systems for the production of a variety of heterologous proteins exist, Fungal expression systems, using e.g. derived from the genus Saccharomyces, Candida, Pichia, Hansenula, Aspergillus or Kluyveromyces are well established (Hollenberg and Gelissen (1997), Current Opinion in Biotechnology 8, 554-560). To circumvent the problem of plasmid instability sometimes encountered in fungi, sequences coding for heterologous proteins are ideally integrated into the fungal chromosome via homologous recombination. Further problems encountered with fungal expression systems are overglycosylation of heterologous proteins and incorrect folding such as incorrect oligomerisation and insufficient ligand incorporation. Expression of heterologous proteins in insect cells—the DNA encoding the heterologous protein can also become integrated into the chromosome via recombination—gets around these problems. However, insect cells lack the ability to produce sialic acid and sialic glycans. Terminal sialic acid residues play divers biological roles in many glycoconjugates. Plants can also be used for the production of recombinant proteins. However, in these heterologous expression system difficulties in extraction and purification prove real bottlenecks.

Mammalian expression system, cultured cells as well as transgenic animals have none of these disadvantages. Recombinant proteins can be produced in cultured mammalian cells either transiently or constitutively (stably). For transient expression of recombinant a vector DNA encoding the recombinant protein is introduced into the cell and in general is not integrated into the cellular DNA. Expression titers of the recombinant protein are at the beginning high. However, since the vector DNA is generally not replicated, the vector DNA becomes diluted with each cell proliferation and hence the expression titer drops. Only rarely a vector DNA or part of the vector DNA illegitimedly recombines with the cellular genomic DNA and the gene encoding recombinant protein is stably integrated into the genome. If the gene encoding the recombinant protein is associated with a selection marker, cells carrying this cassette can be identified and isolated as stably transformed cells. Stable transformants have the advantage that the heterologous proteins are continuously produced. The expression titer is mainly determined by the strength of the promoter construct, the site of integration into the chromosome, the copy number and the type of recombinant protein in question. Many strong promoters are commercially available, however, their transcriptional activity varies depending on the cellular level of the relevant transcription factors and on the chromatin structure at the integration site. For example integration within the scaffod- or matrix attachment regions (S/MAR elements) of chromosomal DNA can augment the activity of promoters—and hence the expression of heterologous genes—and protect them from inactivation by the flanking chromatin. Therefore, it is highly desirable to chose a promoter highly active in a specific cell and to direct integration into an active part of the chromosome. Preferentially a single integration event is desirable, since heterologous genes at low copy number are in general expressed more stable than multicopy genes.

Integration at a single preselected highly active locus can be achieved via homologous recombination. This method, although typically applied to mouse embryonic stem cells, is extremely inefficient in somatic cells of human origin and requires a large scale screening effort. Moreover it is not applicable for most human permanent cell lines when it is desired to completely shut off the expression of a given target gene, because these cell lines are usually polyploid and targeting more than 2 identical loci is hardly feasible. Site specific recombination using recombinases, eg. Cre, flp, C13 and their respective target site (RRS) are a viable alternative (Feng, Y. Q. et al., Journal of Molecular Biology, vol. 292(4), p. 779-785 (1999); Schlake, T. et al., Biochemistry, Am. Chem. Soc., vol. 244(1-2), p. 185-193 (October 2000); Fussenegger, M. et al., Trends in Biotechnology, vol. 17(1), p. 35-42 (January 1999); Groth, A. C. et al., Proceedings of the National Academy of Sciences of USA, vol. 97(11), p. 5995-6000 (May 2000)). With this approach, a plasmid carrying a single RRS can be used to target a single RRS in the chromosome. This method, however, has certain limitations: Namely, it is quite inefficient because the reverse reaction, excision of the plasmid, is an intermolecular recombination and takes place at much higher speed than the integration. Secondly, the whole plasmid including bacterial genes are integrated. To solve the first problem unidirectional was established, e.g. by meains of hetero-specific target sites for both flp and cre. These RRS are recognised by the respective recombinase but a successful recombination requires identical sites and the excision reaction is precluded (Karreman S. et al., Nucleic Acids Res., vol 24(9), p. 1616-1624 (1996); Trinh, K. R. et al., J. of Immunol. Methods, vol. 244, p. 185-193 (2000)). However, the targeting plasmid still has to be integrated into a single favourable position of the chromosome. A large scale screening effort is required to find such rare integrates. These clones often contain more than one copy of the plasmid, the may contain incomplete copies and bacterial sequences care not precluded from integration. These bacterial sequences are recognized by the mammalian cell often leading to inactivation of the targeted region. Alternatively, the targeting cassette may be integrated via retroviral vectors (Karreman S. et al., Nucleic Acids Res., vol 24(9), p. 1616-1624 (1996)). These vectors target active sites within chromosome, only full length cassettes are integrated and the infection dose can be adjusted to create single integration sites. However, expression units flanked by ITRs may also be subject to inactivation. In addition, the use of this system may be restricted by the governmental release agencies to exclude t therapeutic applications of the expressed protein.

### SUMMARY OF THE INVENTION

In view of the above, there is still a need for a method allowing the transformation/conversion of a cell line with an arbitrary gene coding for a product of interest to obtain a high yield recombinant human glycoprotein producing cell, especially for a method without or only little cumbersome screening procedures. It was surprisingly found that cells expressing recombinant glycoproteins with features of human posttranslational modification at high yield are obtainable by first identifying a non-essential highly expressed cellular gene (hereinafter shortly referred to as “starting gene”) in a human or essentially human hybrid cell (hereinafter shortly referred to as “starting cell”); secondly directly replacing the starting gene via homologous recombination with a first functional DNA sequence (e.g. by utilizing an appropriate targeting cassette) containing recombinase recognition sites (RRSs) for site-directed integration and optionally a “place-holder” gene comprising various functional sequences and selecting/isolating a stable clone of this precursor expression cell (functionalized cell); thirdly introducing the gene of interest (from here on called “target gene”) coding for the target gene product (protein) by site-directed integration using a recombinase recognizing the RRSs incorporated with the first targeting cassette; and finally selecting/isolating a stable expression cell capable of producing large amounts of the recombinant protein. Direct replacement of the starting gene with a functional DNA sequence containing a DNA sequence coding for the target gene product is also applicable.

It was moreover found that suitable starting cells for the above method are specific mammalian cells such as human myeloma and hybridoma cells and human heterobybridoma cells (including human-mouse hetero-hybridoma cells such as H-CB-P1), which allow the production of proteins having an essentially human glycosylation pattern.

Using the present invention it is possible to introduce stably any gene encoding a recombinant protein of interest into the specific mammalian cells set forth above. Using the present invention the gene of interest encoding the recombinant protein will become integrated into the locus of a highly expressed cellular gene and preferably in close proximity to a highly active cellular promoter residing in an active part of the chromosome. Using the present invention precursor cell lines of various origin can be created carrying a place holder gene surrounded by RRSs. Using the present invention the place holder gene can be exchanged with the gene of interest, encoding the recombinant protein, by site-specific recombination at the RRSs catalyzed by a suitable recombinase, giving rise to the final high-yield expression cell.

Finally, it was found that the human-mouse heterohybridoma provides for a very distinct human glycosylation pattern.

More specifically, the present invention provides

(1) a process for preparing cells capable of stable high yield expression of a target gene product having essentially human glycosylation pattern which method comprises

(a) selecting a human cell or human hybrid cell (hereinafter “starting cell” capable of stable high yield expression of a starting gene product being non-essential to the starting cell;

(b) screening for the locus of the starting gene product within the genome of the starting cell;

(c1) replacing the gene coding for the starting gene product with a first functional DNA sequence containing one or more recombinase recognition sites (RRS) to obtain a functionalized precursor cell; and

(d) integrating a second functional DNA sequence containing a DNA sequence coding for the target gene product into the functionalized precursor cell obtained in step (c1) by use of a recombinase recognizing the RRSs incorporated with the first functional sequence, or

(c2) directly replacing the gene coding for the starting gene product with a functional DNA sequence containing a DNA sequence coding for the target gene product;

(2) in a preferred embodiment of the method of (1) above the starting cell is an immortalized cell derived from B lymphocytes (preferably is a human-mouse hetero-hybridoma such as hetero hybridoma H-CB-P1 (DSM ACC 2104)) and integration of the functional DNA sequence(s) is effected at a Ig locus (preferably at a rearranged human Ig locus of said cell);

(3) a cell capable of high yield expression of a target gene product obtainable by the method of (1) or (2) above;

(4) a method for preparing a functionalized cell comprising the steps (a) to (c1) as defined in (1) or (2) above;

(5) a precursor cell as defined in (4) above;

(6) a method for high yield expression of a target gene product which comprises cultivating a cell as defined in (3) above; and

(7) a target gene product obtainable by cultivating a cell derived from H-CB-P1.

### DESCRIPTION OF THE FIGURES

FIG. 1: Concept overview, multistep process to create high yield expression cell lines comprising site-specific integration of genes into an IgH locus at frt sites. The IgH locus of H-CB-P1 is represented in the upper graph of FIGS. 1a and 1b. It contains the variable gene promoter followed by a protein leader sequence and specific V, D, and J genes rearranged and positioned next to the enhancer Eμ, the MAR and the Cμ coding sequences. Target sequences for homologous recombination are shown marmorate. Via homologous recombination between the flanking sequence elements “Vhprom” and “Cμ” of vector 1 (targeting vector) and the genomic DNA, first functionalized sequences located between the flanking sequences are introduced into the genomic DNA and a recombinant PBG03 genome is the result. The first functionalized sequence contains frt sites (frtF5 frtF3 and frt wt), an artifical strong promoter CES (FIG. 1a) or no additional promoter (FIG. 1b) upstream of the first expressed gene(hobFc). In addition a blasticidine or hygromycin resistance gene and an ATG deleted neomycin gene are part of the first functionalized sequence. The recombinant genome carries the first functional sequences integrated in the chromosomal DNA.

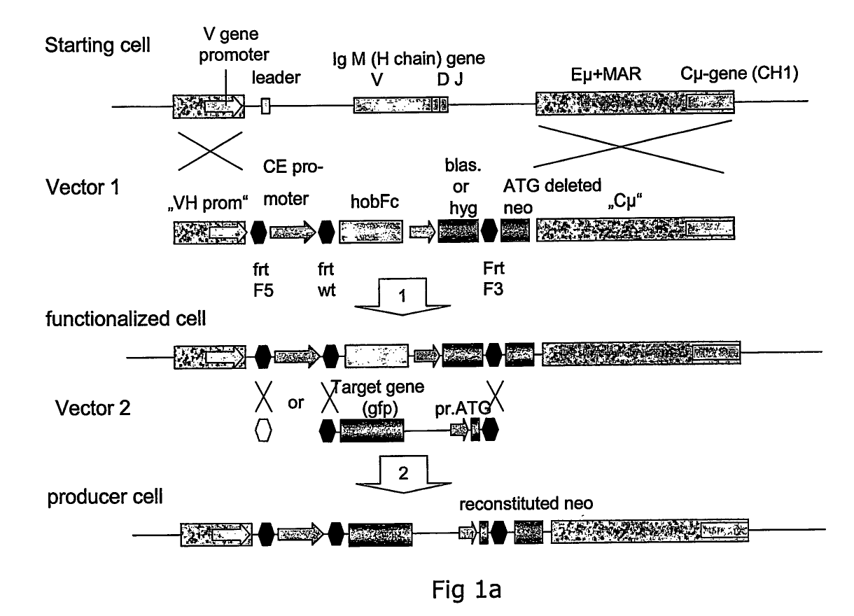

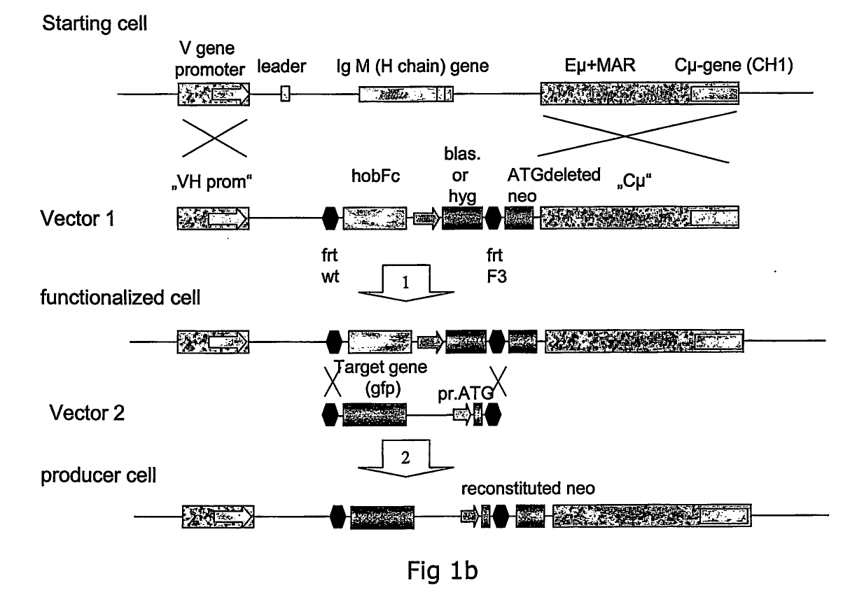

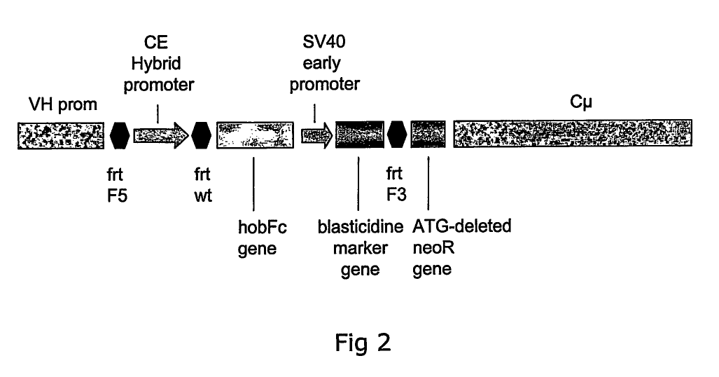

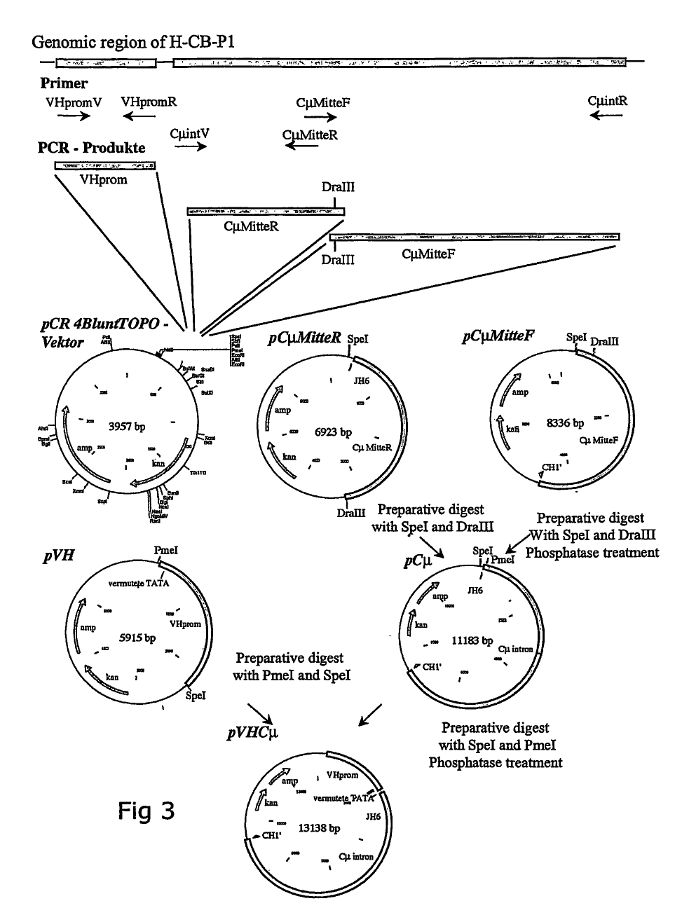

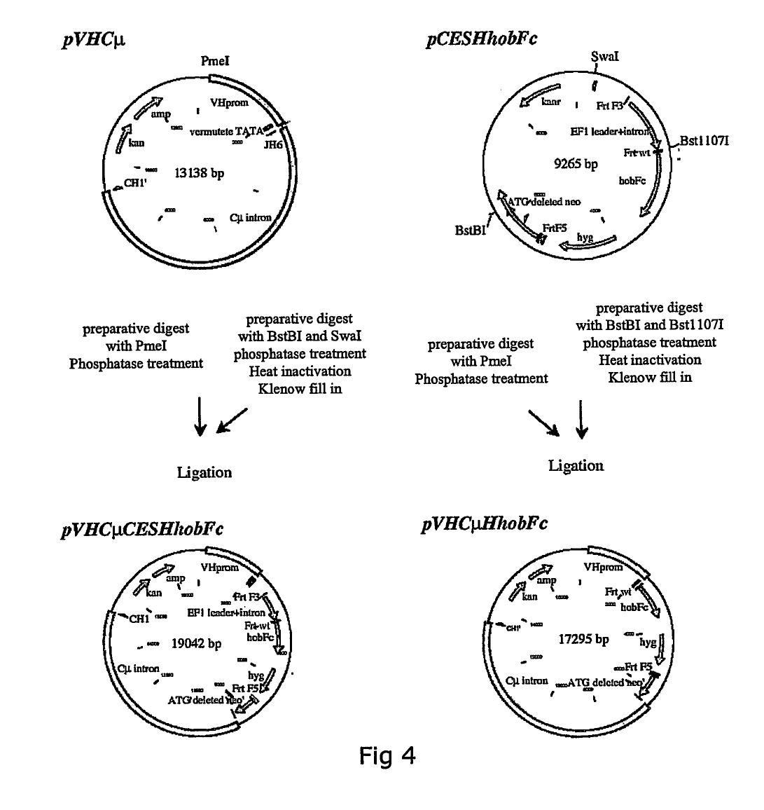

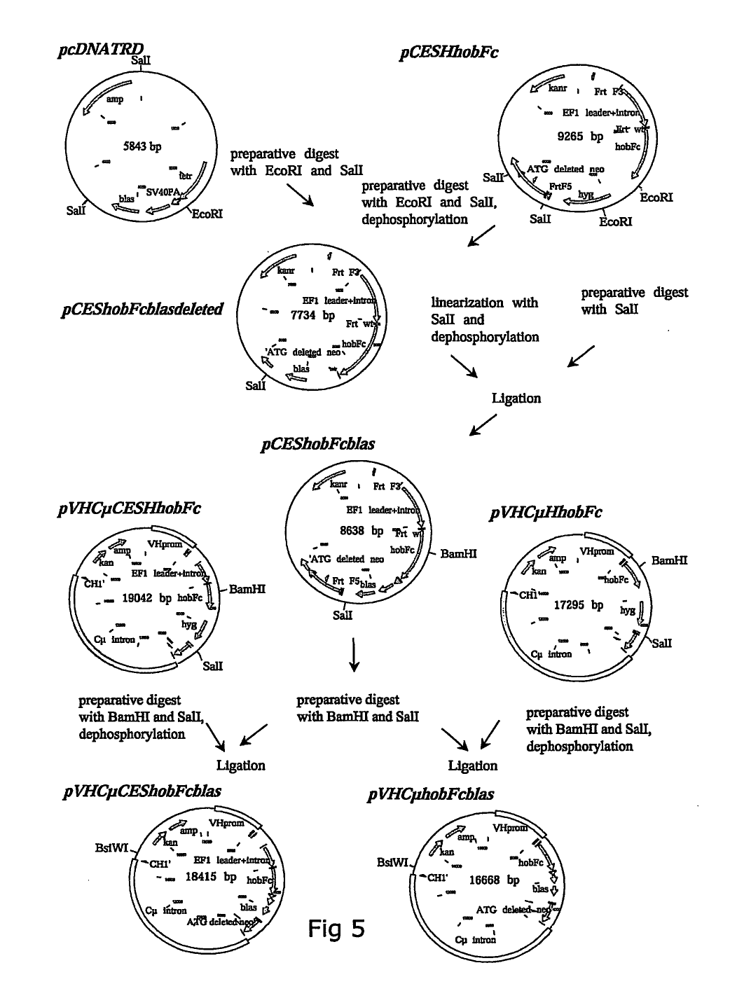

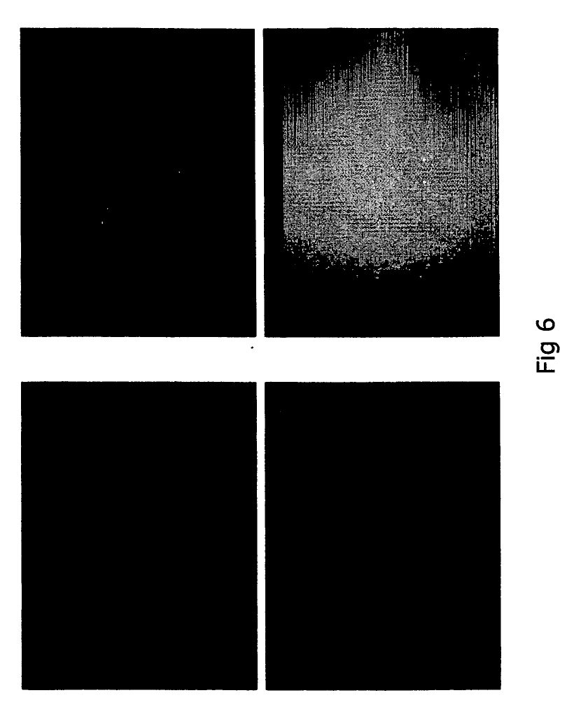

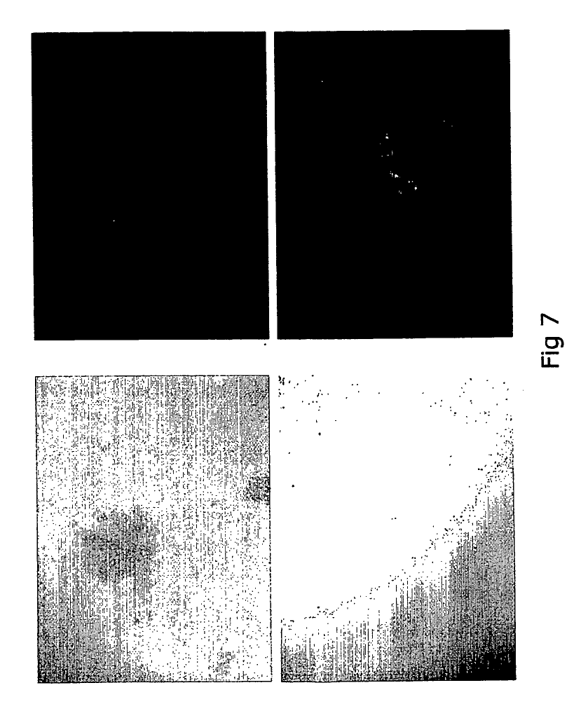

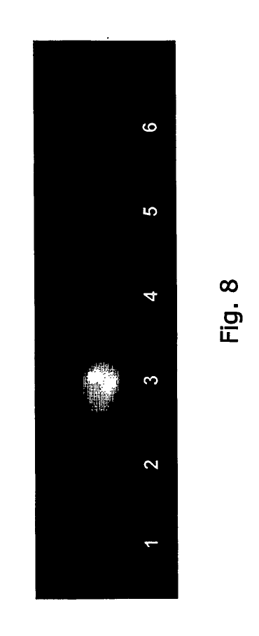

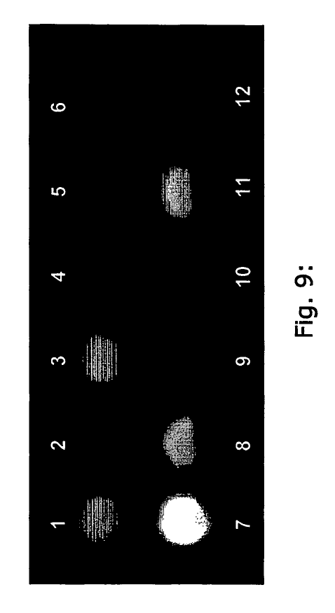

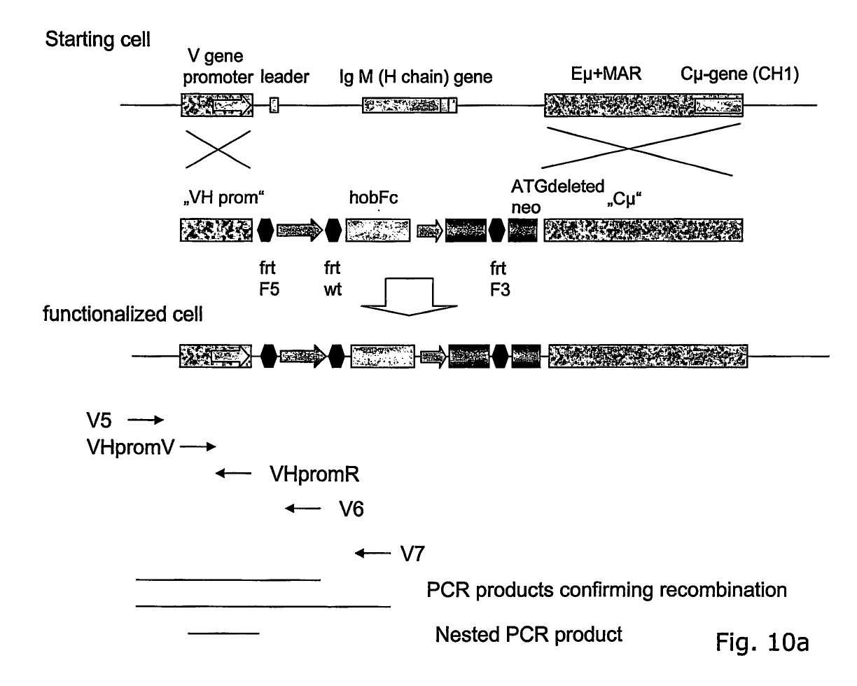

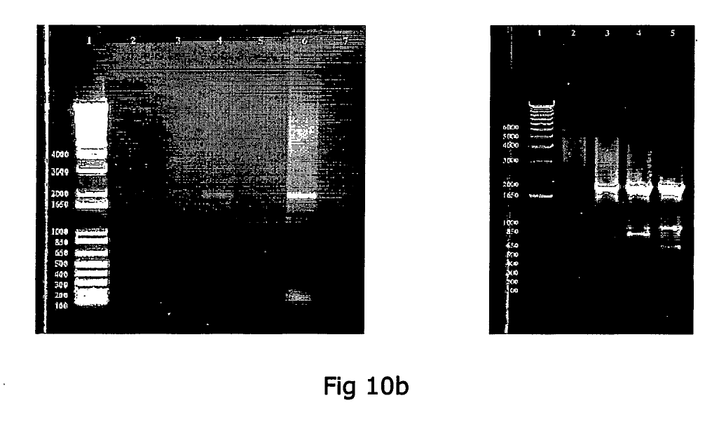

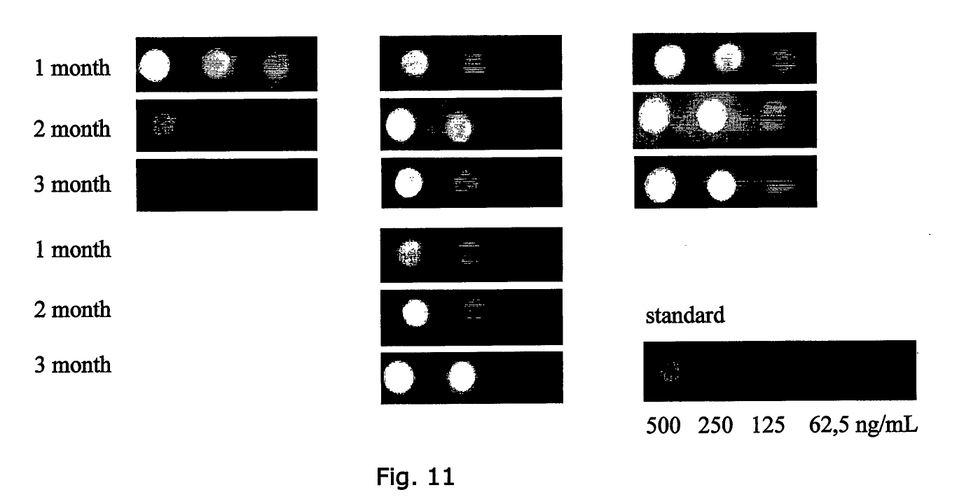

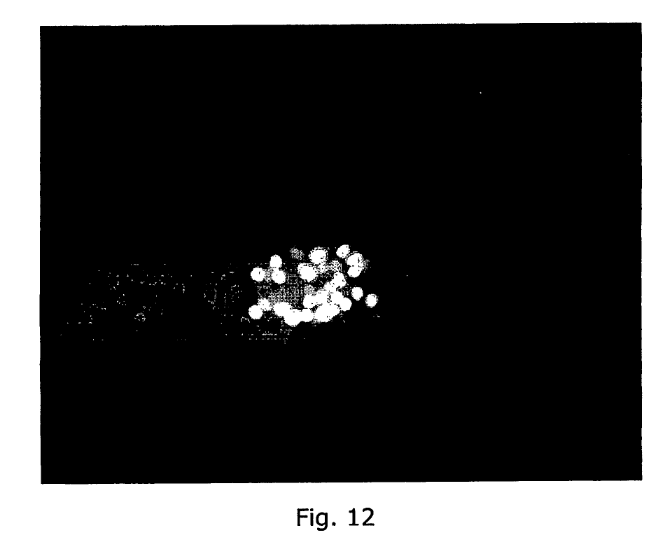

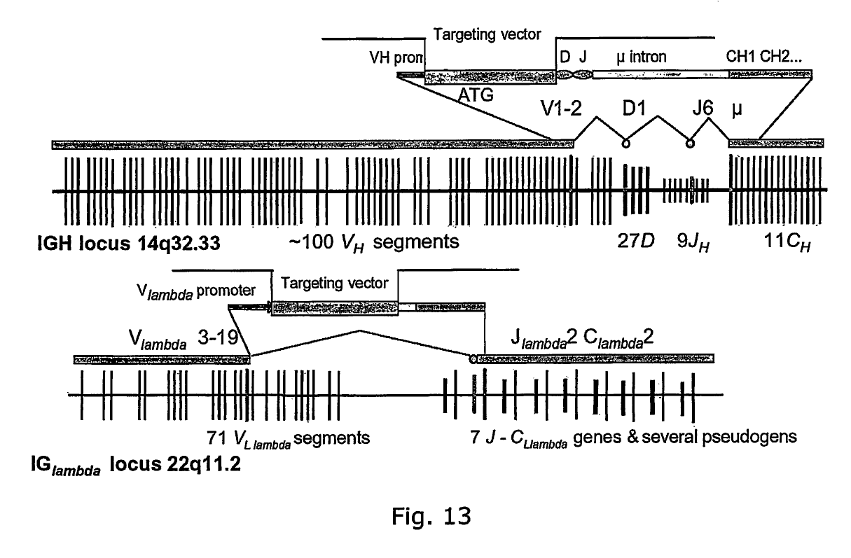

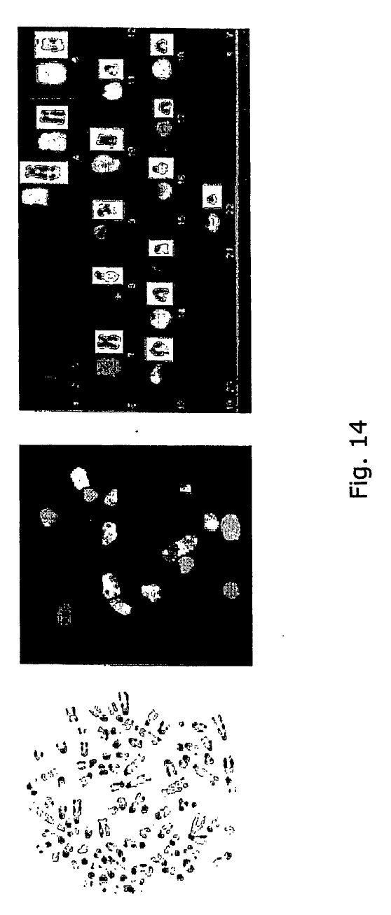

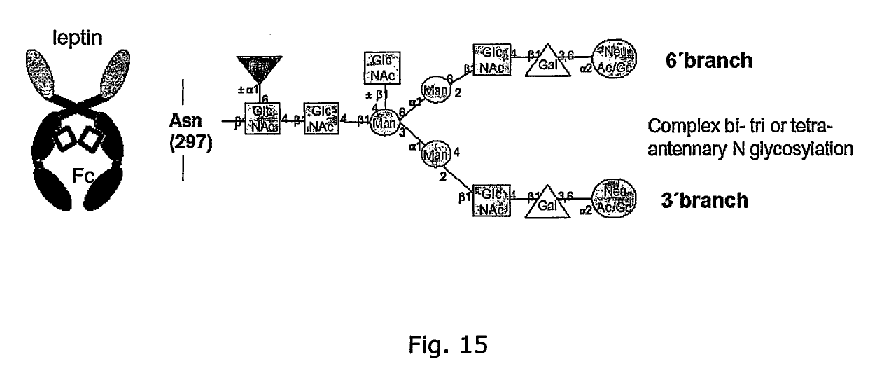

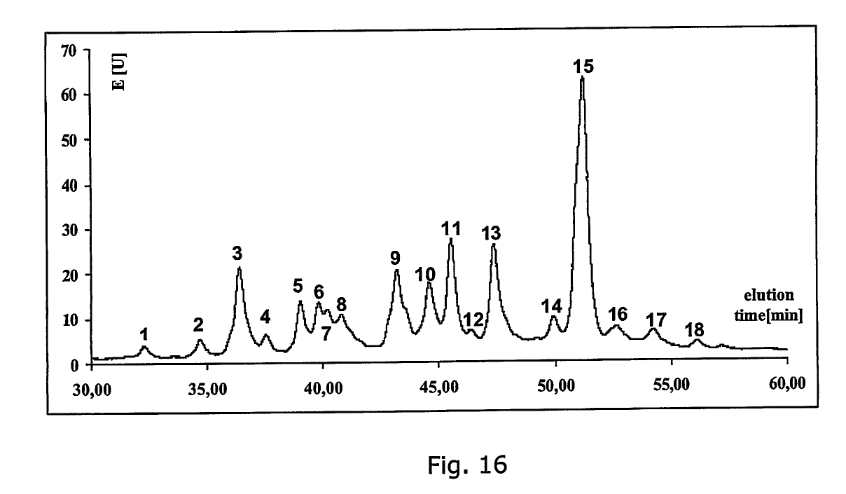

FLP recombinase catalyses recombination at frtF5 and frtF3 or at frt wt and frtF3 sites of the recombinant H-CB-P1 genome and vector 2, the actual gene of interest is introduced and expressed from the artificial or the endogenous VH promoter (FIGS. 1a and 1b, respectively). Parts of the first functionalized sequence located between frtwt and frt F3 sites are replaced by a weak promoter followed by an ATG which after recombination is positioned in the same open reading frame as the ATG-deleted neomycin gene. The resulting genome has lost the hobFc gene and the blasticidin or hygromycin resistence genes and instead has the gene of interest (target gene) and a functional neo gene.

FIG. 2: The endogenous cassette (CEShobFcblas) of the targeting vector. The detailed structure of the endogenous cassette containing an CES promoter, a hobFC fusion gene, a blasticidin resistance gene and a start codon (ATG) deficient neomycin gene is shown. In more detail, the endogenous sequence contains a modified frt site (frtF5) followed by a hybrid promoter structure comprising the early CMV promoter/enhancer elements as well as the first intron of the elongation factor alpha gene. The next element is a frt wildtype site followed by the hobFC fusion gene (hobFC), and the SV40 polyadenylation signal (SV40PA). A weak SV40 promoter controlling the expression of the blasticidin resistance gene follows. The last elements are a modified frt site (frtF3) and next to it the ATG deficient neo gene. Modified frt sites F3 and F5 allow recombination with identical sites but not with wildtype frt sites and F5 or F3 sites respectively. The frt F3 site is positioned to upstream of the neo gene to form a contiguous open reading frame lacking the ATG.

FIG. 3: Cloning strategy for the intermediate vector pVCμ containing the flanking regions Vhprom and Cμ.

The 2 kb VH promoter sequence was amplified from PBG03 cell genomic DNA using forward primer VHpromF (SEQ ID NO:1) and reverse primer VHpromR (SEQ ID NO:2). The PCR product VHprom was cloned into the pCR 4BluntTOPO vector (Invitrogen) and the resulting vector named pVH. The 7.4 kb Cμ region was amplified from genomic H-CB-P1 DNA as two overlapping fragments, namely CμMitteR and CμMitteF. The primers CμintV (SEQ ID NO:3) and CμMitteR (SEQ ID NO:5) give rise to the product CμMitteR and the primers CμMitteF (SEQ ID NO:6) and the reverse primer CμintR (SEQ ID NO:4) produce the fragment CμMitteF. Both fragments were cloned into a pCR 4BluntTOPO vector (Invitrogen) and the resulting vectors called pCμMitteR and pCμMitteF. The full length Cμ sequence was re-established by opening both vectors with the restriction enzyme SpeI and DraIII, and ligating the SpeI-DraIII fragment from pCμMitteR into the opend pCμMitteF. The resulting vector pCμ carries the full-length Cμ region (Cμ intron). The VH promoter sequence and the Cμ sequence were combined in one vector pVHCμ, by digesting both pVH and pCμ with the restriction SpeI and PmeI, and inserting the isolated Vhprom PmeI-SpeI fragment into the phosphatase treated opend vector pCμ.

FIG. 4: Cloning strategy for the targeting vectors pCESHhobFc and pVHCμHhobFc. The targeting vector pVHCμCESHhobFc containging the highly active promoter CES and the hobFC fusion gene was created by ligating an end-filled SwaI-BstBI fragment isolated from pCESHhobFc into the pVHCμ vector that had been digested with PmeI and dephosphorylated. The targeting vector pVHCμHhobFc that has the hobFC fusion gene but no CES promoter, was prepared by ligating an endfilled Bst1107-BstBI fragment isolated from pCESHhobFc into an PmeI digested and dephosphorylated vector pVHCμ.

FIG. 5: Cloning strategy for targeting vectors carrying a blasticidin resistant gene, pVHCμCEShobFcblas and pVHCμhobFcblas.

The plasmid pcDNATRD was used as donor for the blasticidin gene. To delete the hygromicin gene sequences as well as the FRT5 and neomycin sequences from the vector pCESHhobFc, the vector was digested with EcoRI and SalI and dephosphorylated. An EcoI-SalI fragment from pCDNATRD containing the blasticidin resistant gene sequence was ligated into the previously opened pCESHhobFc vector and the resulting plasmid named pCEShobFcblasdeleted. The Frt F5 sequence and the ATG-deleted neomycin sequence were isolated from pCESHhobFc as a SalI SalI fragment and re-inserted into the SalI site of pCEShobFcblasdeleted. ShobFcblasdeleted. The resulting plasmid pCEShobFcblas was used together with pVHCμCESHhobFc to create pVHCμCEShobFcblas. The BamHI-SalI fragment comprising the hobFc sequence and the blasticidin gene was isolated from pCEShobFcblas and inserted into vector pVHCμCESHhobFc opened with BamHI and SalI, giving rise to pVHCμCEShobFcblas. The vector pVHCμhobFcblas was created by ligating a BamHI-SalI fragment containing the hobFc gene and the blasticidin gene into vector pVHCμHhobFc digested with BamHI and SalI.

FIG. 6: Immunostaining of H-CB-P1 clones

H-CB-P1 clones obtained through transfection of H-CB-P1 cells with pVHCμ CESHhobFcblas were immunostained with a Texas Red conjugated anti-human IgG, F_{y} fragments specific antibody isolated from goats or an AMCA conjugated anti-human IgM, Fc_{5μ} specific antibody isolated from goats. The left column shows two H-CB-P1 clones stained with the Texas Red conjugated antibody and visualized with an UV WG filter. In the right column the same clones are shown after staining with the AMCA conjugated anti-IgM antibody and visualized under UV filter WU. Whereas for the clone in the upper panel only IgG staining is evident, staining with both antibodies is present for the clone in the lower panel. The first clone may result from homologous recombination whereas the other clone contains an illegitimate insertion of the functional sequences.

FIG. 7: Direct immunostaining of H-CB-P1 clones and further expansion of clones. It is demonstrated that H-CB-P1 clones could be immunostained without jeopardizing the cells viability and that subsequent expansion of the stained clone was possible. A H-CB-P1 clone cultured for ten days in a 96 well plate was immuno-stained with an Texas Red conjugated anti-IgG antibody. A picture of the clone prior to immunostaining visualized with normal light microscopy, is shown in the top left panel. The same clone immunostained with the Texas Red conjugated anti-IgG antibody is shown in the top right hand panel. The bottom panels show pictures of the well post trypsinisation. No cells can be seen when the picture was taken under a normal light microscope, as shown in the bottom left hand panel. The right hand panel shows the same well examined under UV filter WU. The cells are completely removed from the well and the fluorescent antibody precipitate remained in the well and is not attached to the cell surface.

FIG. 8: Ant-IgM dot blot of cell culture supernatants from induvidual clones Supernatant of the following clones

1: pVHCμhobFcblas-D6; 2: pVHCμhobFcblas-G8;

3: pCEShobFcblas-A3; 4: pVHCμCEShobFcblas-B4;

5: pVHCμCEShobFcblas-D3; 6: pVHCμCEShobFcblas-G8 were spotted onto a membrane and subjected to the ECR staining method. Since the starting cell population homogenously produces IgM, clones with no detectable IgM expression result from inactivation of the IgM H gene mediated by the targeting vector. The clone A3 generated by transfection of pCESHhobFcblas which lacks homologous flanking sequences was unable to target the IgM locus and expresses IgM.

FIG. 9: Anti-IgG dot blot of cell culture supernatants from individual clones. Supernatant from the following clones

1: pVHCμhobFcblas D6; 2: pVHCμhobFcblas D6 (1:2 diluted); 3: pVHCμhobFcblas-G8; 4: pVHCμhobFcblas G8 (1:2 diluted); 5: pCEShobFcblas A3; 6: pVHCμCEShobFcblas B4; 7: pVHCμCEShobFcblas D3; 8: pVHCμCEShobFcblas D3 (1:2 diluted);

9: pVHCμCEShobFcblas D3 (1:10 diluted); 10: pVHCμCEShobFcblas G8;11:hobFc standard 500 ng/ml; 12: hobFc standard 50 ng/ml IgG

were spotted onto a membrane and subjected to the ECR staining procedure using an anti-IgG antibody.

FIG. 10: Detection of homologue recombination via PCR

To test whether a homologue recombination event had occurred between the (targeting) vector and the Ig locus of H-CB-P1 cells, a PCR strategy was applied. Upon recombination the endogenous cassette of the vector containing the CES promoter, the hobFc gene and a resistance gene (hygromycin or blasticidin) followed by the ATG-deleted neomycin gene becomes integrated between the genomic V gene promoter sequences and the enhancer Eμ. The forward primer V5 (SEQ ID NO:7) which binds to the genomic V gene promoter sequences outside of the fragment Vhprom was combined, with primers V6 or V7 (SEQ ID NOs:8 and 9, respectively), which bind specifically within the first functional sequence. The occurrence of PCR products is strictly dependent on co-localisation of both primer binding sequences and hence homologous recombination. To confirm any positives the putative positiv PCR product was used in a nested PCR reaction with the primers VHpromF and VHpromR (SEQ ID NOs:1 and 2, respectively).

a: primer position, b: electrophoretic analysis of PCR products

left: first PCR V5, V7

lane 1: 1 kb ladder (Invitrogen); lane 2: clone pVHCμhobFcblas H6;

lane 3: clone pVHCμCEShobFcblas B10; lane 4: clone pVHCμhobFcblas D4;

lane 5: clone pVHCμhobFcblas D8; lane 6: clone pVHCμhobFcblas E11;

lane 7: neg control H-CB-P1

right: nested PCR

lane 1: 1 kb ladder (Invitrogen); lane 2: neg control H-CB-P1;

lane 3: clone pVHCμCEShobFcblas H6; lane 4: clone pVHCμhobFcblas D4;

lane 5 clone pVHCμhobFcblas E11

FIG. 11: hobFc expression In the absence of selction pressure.

Cells were cultivated for 3 month in the absence of selection pressure. To determine expression cells were seeded at a density of 10⁵ cells/ml. After 24 h cell culture supernatants were harvested and subjected to a westen blot (dot blot) using an anti human Fc antibody. Supernatants were applied to the filter from left to right: undiluted, 1:2 dilution and 1:10 dilution.

Left: clone pCEShobFcblas A3 (random insertion),

Center top pVHCμhobFcblas G8; Center bottom pVHCμhobFcblas G8;

Right pVHCμCEShobFcblas D3.

Expression is stable in clones resulting from homologous insertion of the functionalised sequences including the hobFc gene.

FIG. 12: Generation of a target cell clone using GFP as a model target gene. Clone pVHCμCEShobFcblas D3 (renamed PBG04) was transfected with vector 2 comprising a second fuctional sequence (frtwt, GFP open reading frame and polyadenylation signal, minimal promoter followed by ATG and frtF5) and plasmid pflp comprising a functional expression unit for the flp recombinase (Note: the vector is unable to express GFP in a naive cell because it lacks a functional promoter driving expression). After two weeks of selection with G418 individual stable clones strongly expressing GFP are detectable. GFP expression as well as G418 resistance depends on the homologous recombination event.

FIG. 13: Identification of the rearrangements in the heterohybridoma H-CB-P1 The cDNA for the light and heavy chain genes form H-CB-P1 was sequenced and compared to database sequences. Genomic genes constituting the heavy gene genes were identified by homology with unrearranged genomic sequences. Based on the identification of V1-2, D1, J6 and μ a genomic map of the rearranged locus was constructed. PCR primers were designed using this information, respective fragments extending 5′ to the variable gene V1-2 promoter and containing the D,J, and μ Intron sequences but leaving out the variable gene ATG were amplified and used to construct the targeting vector.

The lambda light chain variable gene V3-19 was identified via homology search as well. This approach was not suitable to identify the constant gene because the locus contains 100% identical gene copies. A PCR based on primers in the intervening sequences between constant region genes allowed to identify J2 and H2 as the genes constituting the rearranged lambda gene of H-CB-P1

FIG. 14: Chromosome analysis of H-CB-P1. GTG Banding, left panel 68-94 chromosomes were fond. The majority were identified as mouse chromosomes Spectral Karyotype Analysis, middle and right panels. Human chromosomes within H-CB-P1 were identified by hybridisation with specifically labeled humanchromosome libraries. 8 intact human chromosomes 4, 5, 7, 10, 14, 17, 18, 22 In addition Chromosome fragments of ch. 4, 8, 9, 10, 11, 14, 16 were identified A hybridization with a probe specific for the human Ig H locus revealed a single IgH locus on the intact chromosome 14

FIG. 15: Schematic representation of a N linked oligosacharide structure of a mammalian glycoprotein.

The leptin-Fc molecule contains two N linked oligosacharides, one on each chain of the Fc domain.

FIG. 16: Aminophase-HPLC of leptinFc

LeptinFc from PBG-04 was generated in roller bottle culture and purified by a generic process including affinity chromatography, gel filtration and membrane filtration. The protein was digested with trypsin and the resulting peptides were deglycosylated by PNGase F digestion. The glycans were labeled with 2-aminobenzamide and separated by HPLC on a Phenomenex Hypersil APS-2-column. Peak numbers represent the fractions which were used in MALDI-TOF-MS analysis.

### DETAILED DESCRIPTION OF THE INVENTION

The present invention provides a method to transform a mammalian starting cell, in particular a human cell or human hybrid cell into a stable high yield expressing cell. To achieve continuous recombinant expression, the gene encoding the recombinant product becomes integrated into the genomic cellular DNA. Expression levels are highly determined by the site of integration of the recombinant gene into the cellular DNA. Therefore, the here presented method comprises the integration of a recombinant gene into a transcriptionally highly active part of the genome of a cell. The gene of interest coding for the recombinant protein can either be under the control of very strong recombinant promoter, or be placed under the control of a highly active cellular promoter by integrating it downstream of the highly active cellular promoter.

In a preferred embodiment of the method (1) of the invention the starting cell secretes the starting gene product, preferably in an amount of at least 0.3 fmol/cell/d of a polypeptide chain (which equals 30 pg/cell/day for a protein of approximately 90 kd) and more preferably in an amount of more than 1 fmol/cell/d (which equals 100 pg/cell/day for a protein of approximately 90 kd). Alternatively, in case the starting cell does not secrete the starting gene product a gene coding for a highly expressed preferably nonessential intracellular or membrane protein or a highly expressed noncoding RNA is selected.

In another preferred embodiment, the starting cell is a primary, immortalized or fusionated cell or a genetic modification thereof. Thus, the starting cell may be selected from primary cells, immortalized cells (e.g. immortalized cells derived from B lymphocytes) or tumor cells or genetic modifications thereof, cell hybrids, cell lines used generally in protein manufacturing such as HEK293, PER.C6 human cell lines created from primary cells via genetic immortalization or fusion with immortal cell lines, preferably it is a human hybridoma or hetero-hybridoma cell (e.g. human-mouse, human-rat or the like) and most preferably is human-mouse hetero-hybridoma H-CB-P1 (DSM ACC 2104; previously referred to as ZIM517).

If the starting cell is a human cell or human heterohybridoma (e.g. as defined above), it is preferred that said hybrid cell or heteor-hybridoma comprises at least one human chromosome and/or is capable of human post-translational modification. It is particularly preferred that the starting gene product is a human gene.

The starting gene product is preferably selected from secreted proteins such as antibodies, cytokines, hormones, enzymes, transport proteins storage proteins, structural proteins, etc. The starting gene product is either known a main product of the chosen starting cell. So stable expression of IgM has been observed for H-CB-P1 or selected in a screening procedure. Screening may be based on individual or combined methods comprising microarray expression analysis, 2D protein gel electrophoresis, quantitative PCR, RNAse protection, northern blot, ELISA and western blot. The power and sensitivity of these individual methods is known to those skilled in the art.

The method of the invention allows the production of any recombinant protein. Preferred target gene products include, but are not limited to, enzymes, in particular proteases, protease inhibitors, hormones, cytokines, receptors or soluble forms thereof (e.g. receptors lacking transmembrane or intracellular domains), full-length antibodies or antibody domains and fusion proteins combining domains of these protein classes.

In a first option of embodiment (1) comprising steps (a), (b), (c1) and (d), the replacement of the starting gene is effected by an one step replacement strategy, wherein the starting cell is contacted with a vector construct containing the first functional sequence, said first functional sequence inactivating and partially or completely replacing the gene coding for the starting gene product. Alternatively, the replacement is effected in a two or multistep strategy, wherein the gene coding for the starting gene product is deleted or inactivated and subsequently contacted with a vector containing the first functional sequence, said first functional sequence being incorporated at the site of the deleted/inactivated starting gene product.

Specific incorporation of the first functional sequence at the location of the starting genes is facilitated by sequences flanking the first functional sequence in the vector which are homologous to the target gene or adjacent sequences. These flanking sequences are obtained either from lambda, cosmid, pac or bac libraries of the starting cell or generated by PCR using starting cell DNA as a template. The percentage of cell clones resulting from specific incorporation of the first functional sequence at the location of the target gene may be further increased by employing a dual selection strategy, where a positive selection marker is contained as part of the first functional sequence and a negative selection marker separated from the first functional sequence by a homologous flank. Homologous exchange allows incorporation of the positive selection marker in the absence of the negative selection marker. Examples for positive selection markers are the hygromycin, blasticidin, neomycin, or glutamin synthetase genes and the HSV tk or the Cytosine desaminase gene are negative selection markers. Markers and methods for their application are known to those skilled in the art.

Cell clones resulting from homologous exchange are identified by the presence of elements of or gene products expressed from the first functional sequence and the inactivation of at least one allele of the starting gene. These cell clones represent the functionalised precursor cell.

The first functional sequence comprises one or more RRS(s) selected from loxP, frt, att L and attR sites of lambdoid phages, recognition sites for resolvases or phage C31 integrase. It is preferred that said recognition sites provide for unidirectional integration, which is achieved, e.g. by modified loxP and frt sites as well as by the (wild-type) recognition sites of ΦC31 integrase. The first functional sequence may further comprise sequences selected from marker sequences, secreted protein genes, promoters, enhancers, splice signals, polyadenylation signals, IRES elements, etc.

To create a producer cell for the target gene product, the functionalized precursor cell (if not already a producer as obtained in step (c2) of second option of embodiment (1), see below), e.g. the PBG03 clone D3 (DSM ACC2577), is subsequently contacted with a second vector containing the second functional sequence. The second functional sequence comprises the target gene and RRS(s) for said recombinases present in the first functional sequence. The second functional sequence further comprises functional sequences selected from promoter sequences, marker sequences, splice donor and acceptor sequences, recombinase recognition sequences differing from RRS of the first functional sequence, etc.

The integration of the second functional DNA sequence is effected by recombinases recognising the RRS with or without accessory proteins (e.g., Cre, Flp, φC31 integrase, resolvase and the like). These recombinase and accessory proteins, mRNA coding for these proteins or viral or nonviral vectors allowing there transient expression are delivered together with, shortly before or after delivery of the second functional sequence.

A pure population of clones containing the second functional sequence at the location of the starting gene is achieved by selection using a reconstituted functional selection marker gene. As an example an inactive ATG deleted selection marker gene introduced with the first functional sequence may be reconstituted by delivery of an active promoter and an in frame-ATG codon with the second functional sequence.

In a second option of embodiment (1) of the invention (comprising steps (a), (b) and (c2)) the gene coding for the starting gene product may directly (i.e. without the provision of the precursor cell) be replaced with a functional DNA sequence containing a DNA sequence coding for the target gene product (hereinafter shortly referred as “third DNA sequence”). Said third DNA sequence may be incorporated by a one- or multi-step strategy as described herein before. The third DNA sequence may further contain functional sequences (such as promoters, markers etc.) as the first and second DNA sequence described herein before. This second option of embodiment (1) of the invention is particularly preferred, if only one target gene product is to be produced so that the generation of the precursor cell is not necessary.

In preferred embodiment (2) of the invention, the starting cell preferably is a human-mouse hetero-hybridoma cell, preferably is hetero-hybridoma cell H-CB-P1 (DSM ACC2104). The integration of the functional DNA sequence is effected at a Ig locus, preferably at one of the human rearranged Ig loci (e.g. heavy chain or light chain (λ or κ)) of the hybridoma cell. The rearranged immunoglobin locus is the genomic sequence surrounding the functional Ig gene (heavy chain λ or κ) modified from the germ line chromosomal configuration during maturation of the B-lymphocyte which gave rise to the hybridoma. The IgH locus Is located at chromosome 14q32.33. In H-CB-P1 this locus is formed by the rearranged and affinity matured VH1-2 gene linked via a D-gene to the J_{H}6-gene linked via the μ-intron to the Cμ sequences (DD 296 102 B3). The sequence of the rearranged VDJ region of the H-CB-P1-IgH locus is provided in SEQ ID NO:12.

It is preferred that the cells of embodiments (3) and (5) of the invention are derived from H-CB-P1 (DSM ACC2104). Furthermore, it is preferred in embodiment (3) of the invention that the target gene product is an antibody. In such case the cell is preferably PBG04 (DMS ACC2577). In the above cells in particular utilized for the expression of antibodies it is feasible that its light chains are inactivated (disrupted) or replaced with a gene coding for same or different target gene product.

Moreover, as indicated before, the target gene products obtainable by expression of a cell line derived from H-CB-P1 possesses a unique essentially human glycosylation pattern.

Glycoproteins for therapeutic application, in particular antibodies are typically manufactured in mammalian cells because posttranslational modifications such as N linked glycans are generated only in mammals and they have a substantial impact on pharmacological features of these proteins. A fully processed N glycan forms a biantennary structure with core fucose and terminal sialic acids (FIG. 15). The majority of proteins, under physiologic conditions, carries only truncated versions of the full structure. The degree at which glycosylation is driven to completion depends on the cell type as well as on culture conditions.

So the degree of sialinisation the addition of the terminal sialic acid to the glycan varies and does rich only 30-40% for antibodies in human blood. However, a high percentage of sialinated proteins increases the half life of a therapeutic protein in blood. The cell lines derived from H-CB-P1, such as PBG04 generate highly sialinated glycoproteins in comparison to CHO and NS0 cells widely used in the manufacture of glycoproteins as can be seen from Example 5. For therapeutic glycoproteins a low content of glycans terminated before the addition of galactose (G0 structures) is advantageous. So G0 Glycoproteins tend to dimerize and the ability of antibodies to mediate complement dependent cytotoxicitly is diminished. The genetic composition of PBG04 allows a more complete processing with a low degree of G0 structures (4.3% on leptin-Fc In a roller bottle process)

Whereas the general biantennary structure is formed by all mammals, some specific structures (linkages between individual sugars) are either specific to, or completely excluded in humans. These structures affect biological features as well. Therefore, it is of advantage to use cells to manufacture glycoproteins for therapeutic applications which provide the necessary enzymes to generate human specific modifications and lack enzymes which are not present in human cells are responsible for atypical linkages. Such cells may be entirely human or contain a subset of human chromosomes. In the latter cells it is important that the human specific glycosylation enzymes dominate over those, not present in humans.

So neuraminic acid may be added as N acetylneuraminic acid or N glycolylneuraminic acid, the latter being the major structure in mouse cells. N glycolylneuranminic acid is absent on glycans form old word monkeys and man. They are immunogenic and may lead to the formation of antibodies against the therapeutic protein.

In addition, mouse cells contain an additional glycosylation enzyme, the alpha 1,3 galactosysltransferase. It mediates the transfer of gal residues to exposed gal residues of the glycan. Such linkage is also found in yeast and as a protection humans have pre-existing antibodies against this structure. Recognition may lead to the formation of immune complexes and kidney damage as a result of treatment. Only 1.3% PBG04 derived leptin-Fc contains alpha 1,3 gal.

A small percentage of human proteins contains bisecting N-acetylglycoseamine. It does not, per se, influence biologic features but the enzyme complex interferes with another one mediating core fucosylation. Often, in proteins with bisecting N-acetylglycoseamine, core fucose is missing, resulting in more efficient binding of the Fc-gamma Receptor and in enhancement of antibody dependent cellular cytotoxicity (ADCC). Therefore cells have been engineered to express (1,4)-N-acetylglucosaminyl transferase III to increase the percentage of non-core-fuccosylated proteins. (U.S. Pat. No. 6,602,684)

A high content of glycoproteins without core fucose can also be achieved in mouse cells. However, these proteins contain the disadvantageous features typical for mouse proteins. A specific hybrid cell of a human a and a mouse with the right chromosomal composition cell can combine the advantageous features of both. Cell lines derived from H-CB-P1 such as PBG04 are such cell lines.

The present invention is further explained by the following examples which are, however, not to be construed to limit the invention.

The cell line H-CB-P1 was deposited at the “Zentralinstitut für Molekularbiologie, Akademie der Wissenschaften der DDR”, Robert-Rössle-Str. 10, Berlin Buch, DDR-1115 as ZIM-0517 on Mar. 16, 1990 and was transferred to the DSMZ, Deutsche Sammlung von Mikroorganismen und Zellkulturen GmbH, Maschroder Weg 13, 38124 Braunschweig, Germany, on Dec. 12, 2000 and here given the depositary number DSM ACC2104. The PBG03 clone D3 (pVHCμCES hobFcblas) was renamed PBG04 and was deposited at the DMSZ as DSM ACC2577 on Sep. 18, 2002.

### EXAMPLES

Materials and Methods

Materials:

DNA Cloning Techniques

Isolation of Genomic DNA: Cells from a T25 cm² flask were trypsinized (see chapter “Trypsinisation” below), the resuspended cell pellet transferred into a 1.5 ml Eppendorf tube and 200 μl PBS added. The tube was centrifuged for 5 min at 13,200 rpm, the supernatant discarded and the pellet re-suspended in 2 ml of solution A. After transfer of the suspension into a Falcon tube (15 ml), 133 μl 10% SDS and 333 μl protease K were added. Either a 3 h incubation at 55° C., or an over night incubation at room temperature followed. The suspension was mixed with 607 μl, 6 M NaCl and vortexed for 15 s before centrifuging it (4300 rpm, 4° C., 20 min). The supernatant was transferred into a Falcon tube (15 ml) and mixed with 2.5 ml 100% Ethanol. A threadlike DNA precipitate formed at the interface and was removed with a pipette tip and re-suspended in ½ TE buffer. The DNA was allowed to completely dissolve at 56° C. before it was stored at 4° C.

PCR: The PCR method was used to isolate genomic DNA sequences (preparative PCR) or to detect certain DNA sequences (analytical PCR).

Preparative PCR: Preparative PCR reactions (50 μl) were set up with the Expand High Fidelity PCR kit (Roche) according to the manufacturer's Instruction (20-30 ng of template, 5 μl of 15 mM Mg Cl₂ buffer (10×), 5 μl dNTP mix, 0.5 μl of each primer (30 nM), 0.5 μl polymerase and filled to 50 μl with water). The PCR products were purified using a QIAquick PCR purification kit (QIAGEN).

Analytical PCR: Analytical PCR reactions (10 μl) were prepared with Taq polymerase kit (QIAGEN) following the manufacturer's instruction (10 ng of template, 1 μl 10× buffer, 0.5 μl dNTP mix, 0.1 μl of each primer (30 μM), 0.1 μl Taq polymerase and filled to 10 μl with water).

The PCR cycling program varied for each product according to the annealing temperature of the primer (see Table 2) and the length of the expected PCR products (determined elongation time and temperature; see Table 1).

TABLE 1
Length of the amplified fragment determines elongation time

| | parameter | | | | | | | | | |
|---|---|---|---|---|---|---|---|---|---|---|
| | length, time | | length, time | | length, time | | length, time | | length, time | |
| | temperature | | temperature | | temperature | | temperature | | temperature | |
| length of | <750 | bp | 1.5 | kb | <3 | kb | >3 | kb | 6 | kb |
| fragment | | | | | | | | | | |
| elongation | 45 | s | 1 | min | 2 | min | 3 min + 15 s | | 4 min + 15 s | |
| time | | | | | | | after each step | | after each step | |
| temperature | 72° | C. | 72° | C. | 72° | C. | 68° | C. | 68° | C. |

TABLE 2
Annealing temperature for primers

| | | |
|---|---|---|
| Primer/SEQ ID NO: | Sequence (annealing temperature [° C.]) | Producer |

| | | |
|---|---|---|
| VHpromF/1 | ATACTAGTCGGCCGCAGGCACATCCACAGTCAC (55) | GIBCO BRL |
| VHpromR/2 | TCCCGGGTATCGATGGAGCTCTCAGGGGATTC (55) | GIBCO BRL |
| CμintV/3 | CATCGATCCGCTACTACTACTACATGG (55) | GIBCO BRL |
| CμintR/4 | CGGCCACGCTGCTCGTAT (55) | GIBCO BRL |
| CμMitteR/5 | AGCTCACCTGGTGCAACT (54) | GIBCO BRL |
| CμMitteF/6 | GACCTAAGCTGACCTAGAC (54) | GIBCO BRL |
| V5/7 | TCCCTC-CAAAAGCTGTAG (52) | TIB |
| V6/8 | ATGGCGGTAATGTTGGAC (52) | TIB |
| V7/9 | CACAAGAATCCGCACAGG (54) | TIB |
| EBVtestR/10 | CCTGATATTGCAGGTAGG (52) | GIBCO BRL |
| EBVtestF/11 | TACCGACGAAGGAACTTG (52) | GIBCO BRL |

In bold: restriction sites

Amplification, isolation and quantification of plasmid DNA: E. coli transformants were grown in a 1 ml, 30 ml or 100 ml culture and plasmid DNA isolated using Mini-, Midi- or Maxi-plasmid purification kits (QIAGEN), respectively. The instruction of the manufacturer were followed. The DNA concentration was determined via spectroscopy, measuring the absorbance at 260 and 280 nm.

Restriction Enzyme Digestion: Plasmid DNA was digested with 1 unit of the appropriate restriction enzyme for 1 μg of DNA, using the buffer and temperature recommended by the supplier (see Table 3). If the analysis required the use of two or more restriction enzymes, the reactions were carried out simultaneous digestion if possible. Otherwise, sequential single digestions were performed with an intervening column purification step (QIAGEN) of the reaction mix.

TABLE 3
Used Restriction Enzymes

| Enzyme | Conc. | Temp. | Buffer | Inact. | Producer | Art. No. |
|---|---|---|---|---|---|---|
| BamHI | 20 U/μl | 37° C. | 2^{a} | 65° C. | BioLabs | R0136L |
| BgIII | 40 U/μl | 37° C. | M | 65° C. | Boehringer | 1175068 |
| BsaBI | 10 //μl | 60° C. | 2 | 80° C. | BioLabs | R0556S |
| BsiWI | 10 U/μl | 55° C. | 3 | 80° C. | BioLabs | R0136L |
| Bst11071 | 5 U/μl | 37° C. | 3 + BSA | 80° C. | BioLabs | R0553S |
| BstBI | 20 U/μl | 65° C. | 4 + BSA | 80° C. | BioLabs | R0519S |
| ClaI | 10 U/μl | 37° C. | H | 65° C. | Roche | 404217 |
| DraIII | 20 U/μl | 37° C. | 3 + BSA | 65° C. | BioLabs | R0510L |
| EagI | 10 U/μl | 37° C. | 3 + BSA | 65° C. | BioLabs | R0505S |
| EcoRI | 40 U/μl | 37° C. | H | 65° C. | Roche | 200310 |
| HincII | 10 U/μl | 37° C. | 3 + BSA | 65° C. | BioLabs | R0103S |
| HindIII | 40 U/μl | 37° C. | B | 5° C. | Roche | 798983 |
| KpnI | 10 U/μl | 37° C. | L | 65° C. | Roche | 899186 |
| Mfel | 10 U/μl | 37° C. | 4 | 65° C. | BioLabs | R0589S |
| NotI | 10 U/μl | 37° C. | 3 + BSA | 65° C. | BioLabs | R0189L |
| PmeI | 10 U/μl | 37° C. | 4 + BSA | 65° C. | BioLabs | R0560L |
| PstI | 10 U/μl | 37° C. | H | 80° C. | Roche | 621633 |
| PvuII | 5 U/μl | 37° C. | 3 + BSA | 65° C. | BioLabs | R0150S |
| SacII | 20 U/μl | 37° C. | 4 + BSA | 65° C. | BioLabs | R0157S |
| SalI | 10 U/μl | 37° C. | H | 65° C. | Boehringer | 567663 |
| ScaI | 10 U/μl | 37° C. | H | 80° C. | Roche | 775266 |
| SmaI | 20 U/μl | 37° C. | 4 + BSA | 65° C. | BioLabs | R0141S |
| SpeI | 10 U/μl | 37° C. | 2 + BSA | 65° C. | BioLabs | R0133L |
| SspI | 5 U/μl | 37° C. | 2 + BSA | 65 | BioLabs | R0182L |
| StyI | 10 U/μl | 37° C. | H | 65° C. | Roche | 85111023 |
| SwaI | 10 U/μl | 25° C. | 3 + BSA | 65° C. | BioLabs | R0604L |
| XbaI | 10 U/μl | 37° C. | H | 65° C. | Boehringer | 674265 |

^{a}specific buffer

End repair of DNA with 5′ protruding termini: To “blunt” 5′ overhangs such as those produced by EcoRI, the digested DNA was treated with the Klenow fragment (Roche) according to the manufacturer's instruction. The endfilling reaction was stopped by a heat inactivation step (65° C., 20 min) and the DNA ethanol precipitated or directly subjected to gel purification.

Dephosphorylation of vector DNA: To prevent self-ligation of linearised vector DNA (see passage “Ligation” below) with compatible ends, DNA was dephosphorylated using alkaline phosphatase (AP) (Roche) according to the manufacturer's instruction. The AP was heat inactivated (65° C., 15 min) and the DNA gel purified (for electrophoresis see passage “Agarose Gel Electrophoresis” below) prior to use in a ligation reaction.

TOPO Cloning: PCR amplification products were cloned into TOPO vectors from Invitrogen. According to the instruction of the TOPO cloning kit, the purified PCR product (0.25-2 μl) was mixed with the salt solution (0.5 μl), water added to reach a volume of 2.5 μl and then the TOPO vector (0.5 μl) added. Following a 30 min incubation at room temperature, the reaction tube was transferred onto ice, 2 μl of the reaction added to “one shot chemically competent E. coli” and the cells incubated for 30 min on ice. The cells were heat-shocked (42° C., 30 s), immediately transferred back onto Ice and 250 μl of room temperature SOC medium added. The transformation reaction was incubated for 1 h at 37° C. with shaking (300 rpm) before the mixture was plated on LB plates containing either Kanamycin or ampicillin. The plates incubated of overnight at 37° C.

Ligation: All ligation reactions were carried out in 10 μl volumes with 0.1-1 μg of dephosphorylated vector and an excess of insert. The reaction contained 2 μl T4 ligation buffer (Gibco BRL) and 1 μl T4 ligase (Roche), and were incubated for two hours at 16° C. or overnight at 4° C. The ligation reaction was transformed into bacteria.

To reduce the level of unwanted non-recombinants, ligations could be postdigested with a suitable restriction enzyme if there was a unique restriction site in the self-ligated vector. Following the digestion, the ligation reaction was ethanol precipitated in the presence of acrylamide (centrifugation at 4° C., 14,000 rpm, 15 min) before the re-dissolved DNA was used again in a transformation reaction

Transformation of competent bacteria: Competent E. coli XL2 (stored at −70° C.) were thawed on ice, mixed with either the ligation reaction (also kept on ice, see passage “Ligation” above) or with 1-100 ng plasmid DNA (re-transformation) and incubated on ice for 20 min. Subsequently the transformation reaction was heat-shocked (30-60 s, 42° C.), the tube returned onto ice and 205 μl SOC medium (free of any antibiotic) added and the reaction incubated shaking (300 rpm) at 37° C. for 45 min. The transformation reaction was plated on LB plates containing an antibiotic (either kanamycin (40-60 μg/ml) or ampicillin (50-100 μg/ml)) and incubated overnight at 37° C. The bacterial colonies were counted and the efficiency of the transformation reaction calculated.

Agarose Gel Electrophoresis: DNA fragments were separated according to their length on 0.7-1.5% agarose gels. The agarose was dissolved in 1×TAE buffer and 2 μl ethidiumbromide/100 ml agarose added. When the agarose had dissolved, it was poured into a tray and allowed to set. The DNA sample was mixed with the loading buffer Orange G, loaded onto a horizontal gel and run at 40-90 V with 1×TAE as running buffer. The DNA/ethidium bromide complexes were visualized under UV light.

Gel Purification of DNA Fragments: The DNA was separated on an agarose gel (40-80 V) and DNA bands (visualised under UV light) of interest excised with a scalpel. Using a QIAquick gel extraction kit (QIAGEN), the DNA was extracted from the agarose block according to the manufacturer's instructions.

Cell Culture

Trypsinisation: Adhesive cells were harvested using trypsin. First the culture medium was removed and the cell monolayer washed with citrate buffer (pre-warmed to 37° C.). A small amount of trypsin was added directly to the cell monolayer and incubated for 3-5 min at 37° C. Trypsinisation was stopped by addition of PBG 1.0 medium supplemented with 5% FCS (see Table 4). The cell suspension was transferred into a Falcon tube (50 ml) and centrifuged for 10 min (800 rpm, 30° C.). The cell pellet was re-suspended in fresh medium and the cells used for electroporation or for further passaging.

TABLE 4
Volumes of citrate, trypsin and PBG 1.0 supplemented
with 5% FCS used for the trypsinisation of the
cells growing in different flasks

| Tissue culture | Citrate | Trypsin | PBG 1.0/5% |
|---|---|---|---|
| flask | buffer (ml) | (ml) | FCS (ml) |
| T25 | 1.0 | 0.5 | 4.5 |
| T85 | 2.0 | 1.0 | 9.0 |
| T180 | 5.0 | 2.0 | 8.0 |

Counting of Cells

After the cells had been trypsinized they were counted in a Neubauer chamber (haematocytometer). A small volume of the cell suspension was introduced into the chamber and the chamber placed under a microscope. Only cells within one of the four squares of the chamber were counted, the cell number multiplied with the factor 10⁴ to obtain the number of cells per ml. To differentiate between vital and dead cells, the cells were stained with trypan blue prior to counting. Dead cells appeared blue whereas vital cells did not take up the dye.

Transformation

Electroporation of H-CB-P1 Cells: In a standard electroporation reaction 10 μg of linearised plasmid DNA were used. The culture medium was removed and the H-CB-P1 monolayer washed with citrate buffer and trypsinised (see passage “Trypsinisation” above). The cell pellet was re-suspended in Opti-MEM (pre-warmed to 37° C.) to obtain 3×10⁶ cells per ml. A volume of 700 μl of the cell suspension was transferred into the electroporation cuvette (peqLab; EQUBIO 4 mm) and the linearised DNA (10 μg) added. The cells were electroporated at 250 V, 1500 μF and immediately afterwards transferred into T75 bottles containing pre-warmed PBG 1.0 medium supplemented with 5% FCS, and incubated at 37° C. and 5% CO₂.

Selection

Selection of H-CB-P1 Cells: The electroporated H-CB-P1 cells were cultured for two days at 37° C. and 5% CO₂. On day 2, the culture medium was removed and non-adhesive cells harvested by centrifugation of the culture medium. 1 ml of the culture medium (supernatant) was frozen for later examination of transient expression. The trypsinized cell monolayer and the cells harvested from the culture medium by centrifugation were combined and pelleted. Cells were re-suspended in PBG 1.0 medium supplemented with 5% FCS to obtain dilutions of 1×10⁶, 1×10⁵ and 1×10⁴ cells/ml. Each dilution was supplemented with 5 μg/ml or 10 μg/ml blasticidin or with 200 μg/ml or 400 μg/ml hygromycin. The selection medium was changed on days 4, 7 and 10, and the growth of the H-CB-P1 clones controlled via microscopy. Between days 8 and 10 vital clones were visible by eye. On day 13 clones were harvested by trypsinisation, the cell pellet suspended in PBG 1.0 medium so that when cells were seeded into 96 well plates, a well contained either 5 cell or 1 cell. The selection pressure (either 5 or 10 μg/ml blasticidin or 200 or 400 μg/ml hygromycin) was maintained throughout. On day 10 the cells were immuno-stained to differentiate between positive and negative cell clones. The cell culture medium was removed and replaced with standard medium supplemented with a fluorecently labeled antibody (2 μg/ml) recognizing the recombinant protein produced by the cells. The antibody suspension was left for 4 h on the cell monolayer before it was replaced with OptiMem 1 supplemented with 5% FCS. The cell monolayers were examined under a fluorescent microscope. When Texas red conjugated antibodies were used, the microscopy was done with a UV filter (WG) that is transmissible for 470-480 nm spectra. The excited Texas red labeled antibodies emit light in the spectra of 590 nm. A UV filter (WU) transmissible for spectra of 330-355 nm was used for visualization of antibodies conjugated to AMCA. Emission of excited AMCA occurred in the blue spectra (420 nm). Only clones that were large and strongly fluorescent were considered. Was there only a single clone in one well, the cells were further expanded. Was there more than only one clone in a well, the individual clones (cells) were picked with a microcapillar and transferred into a new 96 well plate for further expansion (see the following passage “Mircopillary Picking”).

Microcapillary Picking: The microcapillary picking device used a capillary attached to a movable arm that was controlled via a joystick. The microcapillar and the arm were inside the hood wheras the joystick was controlled from outside. When an interesting clone was identified via the immunostaining technique (described in section “Selection of H-CB-P1” above), the microcapillary was placed over the clone, negative pressure induced within the capillary through a vacuum pump and the cell pile of interest sucked into the microcapillary. The arm was moved over the fresh well of a 96 well plate and the cells therein ejected.

Cryoconservation: For long term storage cells trypsinized cells were re-suspended at 1×10⁶ to 1×10⁷ cells/ml. The cells were pelleted by centrifugation (700 rpm, 10 min) and the supernatant removed. The cells re-suspended in cold pre-conditioned medium (900 μl), a cryo-vial filled with 180 μl DMSO and 720 μl FCS, and the 900 μl cell suspension transferred into the DMSO/FCS solution. The cryo-vial was stored for 24 h in a special freezing container to freeze the cells gently. For long term storage the cryo-vials were transferred into liquid nitrogen tanks (storage at −196° C.).

Detection of Protein Products

EC-Western-Blot (Enhanced Chemiluminscence): For detection of hobFC antibodies 20 μl of cell culture supernatant were mixed with 10 μl 5% SDS and incubated for 2 min at 97° C. Was the cell culture medium expected to contain IgM antibodies, the culture medium was not treated. A membrane (Amersham-Pharmacia; Hybond-P) was first rinsed in methanol (1 min), washed three times in water (1 min), and then soaked in plot transfer buffer before placing it on a piece of 3 MM paper also soaked with plot transfer buffer. 5 μl of the pre-treated (hobFC antibodies) or 5 μl of the untreated (IgM antibodies) culture medium were spotted onto the membrane and incubated for 1 min. The membrane was placed in blocking buffer, incubated for half an hour under shaking and three-times washed for 5 min in T-PBS. The blocked membrane was placed in a detecting antibody solution (for IgG 1:2000 and for IgM 1:5000) and incubated for 2 h. Three more washes, of 2, 5 and 10 min in T-PBS buffer followed. The membrane was placed in developer (Amersham-Pharmacia, ECL) and incubated for 1 min. Finally it was drained and placed onto 3 MM paper and wrapped in cellophane to prevent drying. The light emission was observed in a dark room.

### Example 1

### Preparation of a Targeting Vector Specific for the IgM Region of H-CB-P1 Cells

The recombinant gene was to be inserted into the IgM sequence region because it is well-known that antibodies are highly expressed and secreted proteins. The final targeting vector hence required sequences that had 100% homology to the targeted genomic IgM sequences. For the basic targeting vector pVHCμ the VH region of 2 kb and the Cμ intron region of 7.4 kb in length were chosen (see FIG. 3). Both regions were isolated as PCR fragments using polymerases with proofreading activity (proofstart polymerase (Qiagen)), subcloned into a pCR 4BluntTOPO vector (Invitrogen) and finally combined in one vector named pVHCμ (see FIG. 3).

Preparation of plasmids pVHCμCESHhobFc and pVHCμHhobFc: The basic targeting vector pVHCμ does not have an endogenous cassette yet. The endogenous cassette containing the CE promoter, the place holder gene hobFc, three FRT recombination sites as well as a hygromycin resistance gene and an ATG deleted neomycin gene was isolated from the vector pCESHhobFc as a BstDI-SwaI fragment. The fragment was then endfilled with Klenow polymerase and ligated into the basic targeting vector pVHCμ that had been digested with PmeI and dephosphorylated. The resulting targeting vector was called pVHCμCESHhobFc.

A second vector pVHCμHhobFc that lacked the CES promoter construct but contained all the other parts of the endogenous cassette was also constructed. A BstBI-Bstl107I fragment was isolated from pCESHhobC and endfilled. The Bstl107I restriction site in pCESHhobFc is immediately upstream of the frt wt site followed by the hobFc gene and thus the Isolated Bstl107I-Bstb1 fragment lacks the promoter. The fragment was ligated into a pVHCμ vector previously digested with PmeI and the resulting vector was pVHCμHhobFc. In both vectors pVHCμCSHHobFc and pVHCμHhobFc the active resistance marker gene of the endogenous cassette was the hygromycin resistance gene. An alternative set of vectors that contained a blasticidin gene instead of the hygromycin gene was also created.

Construction of pVHCμCSHhobFcblas and pVHCμhobFcblas: To create targeting vectors with the blasticidin gene as marker gene in the endogenous cassette, the vector pcDNATRD was used as donor for the blasticidin gene. The first step involved the exchange of the hygromycin gene with the blasticidin gene. To this end the blasticidin gene was Isolated from pcDNATRD as an EcoRI-SalI fragment. The endogenous cassette vector pcESHhobFc was also opened with EcoRI-SalI and thereby the hygromycin gene was removed as well as part of the ATG-deleted neomycin gene. The fragment containing the blastidin gene was ligated into the opened pCESHhobFc and the resulting vector named pCEShobFcblas deleted.

The second step was the re-insertion of the coincidentally removed ATG-deleted neomycin gene. For that a fragment encompassing the ATG-deleted neomycin gene was Isolated from pCESHhobFc and inserted into the opened vector pCEShobFcblasdeleted. The resulting vector was named pCEShobFcblas. This vector now served as donor vector for the blasticidin gene for plasmids pVHCμCSHhobFc and pVHCμHhobFc. A BamHI-SalI fragment was Isolated from pCESHhobFcblas and inserted into a BamHI-SalI opened vector pVHCμCESHhobFc, giving rise to pVHCμCEShobFcblas. To create the control vector pVHCμhobFcblas lacking the CES promoter, the vector pVHCμHhobFc was also digested with BamHI-SalI and again the BamHI-SalI fragment isolated from pCEShobFcblas ligated into it.

### Example 2

### Selection of hobFc Clones

Electroporation: H-CB-P1 cells were electroporated with plasmids pVHCμCshobFcblas, pVHCμhobFcblas, pVHCμHhobFc and pCShobFcblas. In order to determine the transfection efficiency, cells were transfected with plasmid pGFPN1VA and as mock control, cells were electroporated with a water sample. The transfection efficiency was found to be at approximately 20%. On day 2 post-electroporation depending on the transfected plasmid either hygromycin or blasticidin was added to the culture medium. When mock-transfected cells were all dead, cells from the other transfection reactions were harvested and re-seeded at a density of either 1 cell or 5 cells per well into a 96 well plate. Cells were continued to be cultured with medium supplemented with the appropriate antibiotic.

To optimise the selection conditions cells were seeded at 10⁴, 10⁵ or 10⁶ cells/20 ml into T75 flasks two days post-electroporation. The medium was supplemented with either 5 or at 10 μg/m blasticidin, or 200 or 400 μg/ml hygromycin. On day 14 the number of clones per cm² was determined. The highest number of clones was obtained in flasks seeded with at 1×10⁶ cells that had been transfected with the plasmids lacking the CES promoter (pVHCμhobFcblas). Three times fewer clones were obtained when cells had been transfected with plasmids carrying the CES promoter. Furthermore these clones did grow less well as those without the CES promoter.

The effect antibiotics have on the selection of protein producing clones: Following the expansion of the clones in T75 flasks, the clones were trypsinized and re-seeded into 96 well plates at 5 cells/well. The culture medium contained either 5 or 10 μg/ml blasticidin or 200 or 400 μg/ml hygromycin. On day 10 post-seeding, cells were stained with Anti IgG antibodies conjugated with Texas red and AMCA labeled antibodies against IgM. The results obtained with cells cultured with blasticidin showed that with the higher antibiotic concentration far fewer positive clones were obtained. When 10 μg/ml blasticidin were used, 100% more hobFC negative clones were observed compared to 5 μg/ml. However, only the first three rows of the 96 well plate containing cells cultured with 10 μg/ml blasticidin were counted and the result for the entire plate was extrapolated wheras all rows of the 96 well plate containing cells cultured with 5 μg/ml blasticidin where counted.

Advantage of selection with fluorescently labeled antibodies: Using the immunofluorescent staining technique large numbers of clones can be screened rapidly. The results obtained with the direct immuno-technique were a first indication for correct integration of the targeting sequence into the genomic DNA. Non-expressing clones were easily detected with this technique. The immunofluorescent staining method is technically easy, lower concentrations of antibodies are required than with the methyl-cellulose staining technique, and the proliferation of cells is not impaired. Without any noticeable damages, cells could be immuno-stained before subsequent trypsinization or microcapillary picking to transfer cells into new culture vessels.

### Example 3

### Detection of Homologous Insertion into the IgM Locus

For the design of the targeting vector the rearranged immunoglobulin heavy chain locus was assembled based on the cDNA sequence of the antibody and human genome sequence information. To ensure that the production of IgM was disrupted by the targeting approach, the complete leader sequence including the ATG, the V, D and J genes were omitted from the targeting vector and deleted from the genome via a single homologous recombination event. Hence the replacement type targeting vector contained isogenic sequences from the IgM locus to allow the directed cross over as well as the hobFc gene, blasticidin and ATG deleted neomycin resistance genes. Since only one rearranged active IgM locus on chromosome 14 is present in the starting cell line H-CB-P1, the homologous recombination event completely abolishes IgM expression which is detected by fluorescent antibody staining and supernatant immunoblotting using an anti-IgM antibody.

Western Blot (Dot Blot) for IgM and IgG: The dot blot technique was used to verify the results obtained with the direct immuno-staining technique. The supernatant (medium) of these clones was examined for presence of IgM and IgG. If supernatant was found to be IgM negative as well as IgG positive, it was concluded that a homologous recombination event had taken place.

PCR for detection of integrated targeting sequences: To verify that the targeting cassette had become integrated at the IgM locus PCR reactions were set up with the forward primer V5 (SEQ ID NO:7) and reverse primers V6 (SEQ ID NO:8) or V7 (SEQ ID NO:9), and genomic DNA isolated from cell clones as template. Primer V5 binds to the genomic V gene promoter sequences outside of the fragment Vhprompresent in the targeting vector, reverse primer V6 binds within the CES promoter sequences (and hence was only used for cells transfected with plasmids carrying the CES promoter) and primer V7 bind within the hobFc gene. Using the described primer combinations the occurrence of PCR products is strictly dependent on co-localisation of both primer binding sequences and hence homologous recombination. To increase the sensitivity of this PCR assay, first-round PCR products were used as templates in nested PCR reactions with primers VHpromF (SEQ ID NO:1) and VHpromR (SEQ ID NO:2). Finally nested PCR products were subjected to an enzyme restriction digest with HincII and DraI to confirm that obtained sequences were correct. Passing on all these assays, PBG03 clones H6 (pVHCμhobFcblas), D4 (pVHCμhobFcblas), E11 (pVHCμhobFcblas) D3 (pVHCμCEShobFcblas) and G8 (pVHCμhobfcblas) had integrated the targeting sequence correctly. Clone D3 (pVHCμCEShobFcblas) was renamed PBG04 and deposited at the German Collection of Microorganism and Cell Cultures (DSMZ).

### Example 4

### Recombination to Generate a Target Cell Clone

Clone pVHCμCEShobFcblas D3 (PBG04) was transfected with vector 2 comprising a second functional sequence (frtwt, GFP ORF and polyadenylation signal, minimal promoter followed by ATG and frt5) and plasmid pflp comprising a functional expression unit for flp recombinase using the transfection reagent effectene (Qiagen). Vector 2 does not contain a promoter driving the GFP expression unit. The functionalised cell (PBG04) is sensitive to Geneticin selection from 200 μg/ml. Vector 2 misses a neomycin resistance gene sequence which could confer resistance to Geneticin. As expected no green fluorescence was detectable 1-4 days after transfection. After two weeks of selection with Geneticin individual stable clones strongly expressing GFP were detectable. GFP expression depends on integration in direct proximity to a functional promoter. Geneticin resistance is dependent on reconstitution of the neo resistance gene present in the cell line by the ATG from vector 2. We conclude that in all cases vector 2 has replaced sequences between the frtw and frt F5 sites and functionally linked the CES promoter and GFP as well as the ATG deleted neomycin gene with the ATG.

### Example 5

### Studies of the Glycosylation Pattern of Leptin Fc

Leptin Fc from PB604 was generated in roller bottle culture and purified by a generic process including affinity chromatography, gel filtration and membrane filtration. The protein was digested with trypsin and the resulting peptides were deglycosylated by PNGase F digestion. The glycans were labeled with 2-aminobenzamide and separated by HPLC on a Phenomenex Hypersil APS-2-column (FIG. 16). MALDI-TOF-MS (BRUKER BIFLEX™) was used with the desialylated, labelled samples to further characterize the respective fractions shown in Table 5.

The single N-glycosylation site on Fc carries complex oligosaccharide structures which are sialylated at 37%, a rate close to average sialylation on antibodies in human blood. Sialic acids were further characterized by sialidase treatment, DMB labelling and separation on a Bischoff Hypersil-ODS-column and compared with the Sialic Acid Reference Panel (Oxford GlycoSciences). Typically, N-acetylneuraminic acid was found. Only 2% were represented by N-glycolylneuraminic acid, the dominating form in mouse myeloma cells, which was shown to be immunogenic (Noguchi, A. et al., J. Biochem, 17(1):p. 59-62 (1995)). Alpha 1,3 Gal structures, which are not made in human cells and are known to increase clearence via preexisting antibodies, were only found in 1.3% of the glycans. The above findings are summarized in Table 6.

TABLE 5
Identified oligosacharides as the result of
Results of MALDI-TOF-MS analysis of fractions
from the Phenomenex Hypersil APS-2-column

| Peak | Area (%) | Monoisotopic mass (m/z) | Proposed structure |
|---|---|---|---|
| 1 | 1.2 | 1256.9 | Man3HexNAc1 |
| 2 | 1.4 | 1402.9 | Man3HexNAc1Fuc |
| 3 | 8.0 | 1378.4 Main | High Man5 |
| 4 | 1.6 | 1377.9 | High Man5 |
| | | 1419.0 | Man3HexNAc1Hex1 |
| 5 | 4.3 | 1606.5 | Man3HexNAc2Fuc |
| 6 | 3.1 | 1565.5 | Man3HexNAc1Hex1Fuc |
| 7 | 2.7 | 1540.7 Trace | HighMan6 |
| | | 1581.7 Main | Man5HexNAc |
| 8 | 4.7 | 1540.5 | HighMan6 |
| | | 1581.5 Main | Man5HexNAc |
| | | 1622.7 | Man3HexNAc2Hex1 |
| 9 | 9.3 | 1768.8 | Man3HexNAc2Hex1Fuc |
| 10 | 6.6 | 1743.8 Main | Man5HexNAc1Hex1 |
| | | 1784.9 Trace | Man3HexNAc2Hex2 |
| 11 | 8.7 | 1784.7 | Man3HexNAc2Hex2 |
| 12 | 1.6 | 1784.5 | Man3HexNAc2Hex2 |
| | | 1889.7 | Man5HexNAc1Hex1Fuc |
| | | 1930.7 Trace | Man3HexNAc2Hex2Fuc |
| 13 | 10.9 | 1930.8 | Man3HexNAc2Hex2Fuc |
| 14 | 3.0 | 1664.5 | Bi − 2AB |
| | | 1946.9 Trace | Man3HexNAc2Hex3 |
| | | 2093.0 | (Bi + Gal*) |
| | | | Man3HexNAc2Hex3Fuc |
| | | | (Bi + Fuc + Gal*) |
| 15 | 25.7 | 2150.0 | Man3HexNAc3Hex3 |
| 16 | 3.2 | N.D. | |
| 17 | 2.8 | N.D. | |
| 18 | 0.9 | 2031.2 | Tri − 2AB |
| | | 2514.2 | Man3HexNAc4Hex4 |

TABLE 6
Summary of specific features of N linked Oligosacharides from proteins isolated
from human blood, hamster CHO, mouse NS0 cells or the heterohybridoma PBG-04

| Feature | Impact | PBG-04 | CHO | NS0 | human |
|---|---|---|---|---|---|
| Sialylation | ↓ proteol. Sens./Clearence | 37% | variable | variable | 35-40 |
| N acetyl- | wanted | 98% | high | low | 100% |
| N glycolyl- | immunogenic | 2% | low | high _{(>50%)} | no |
| 2-6 linkage | unknown | no | no | no | variable |
| 1-3 alpha gal. | Preexist. Ab: clearence | 1.3% | variable | high | no |
| Bisecting GlcNAc | ? → ↓ core fucosylation | no | no | no | 10% |
| No core fucose | ↑ ADCC, Fcγ-binding | 60% | 5% | 10-50% | 5% |
| G0-structures | ↑Dimerisation, G2→↑CDC | 4.3% | variable | variable | low |

### Example 6

### Preparation of a Targeting Vector for the Light Chain Lambda Locus of PBG04

The structure of the rearranged lambda chain locus was identified by alignment of the known cDNA of the lambda gene with human genomic sequences. The gene consists of a variable gene, J and H segment already joined together. V3-19 was identified as the variable gene.

This approach was not suitable to identify the constant gene because the locus contains 100% identical gene copies. A PCR based on primers in the known leader sequence and in the unique intervening sequences between constant region genes. Primers V81, V83 (SEQ ID NOs:13 and 14, respectively) gave a correctly sized PCR product which showed the expected restriction pattern and therefore allowed to identify J2 and H2 as the genes constituting the rearranged lambda gene of H-CB-P1 (SEQ ID NO:21). Based on this information the sequence of the rearranged locus was proposed and a targeting vector was constructed.

A 5′ flank upstream of the coding sequences of the variable gene V3-19 was created with Primers V89 and V94 (SEQ ID NOs:15 and 18, respectively) using Provestart Polymerase (Qiagen). A 4 kb fragment was cloned in pPCR4blunttopo (Invitrogen). A 3 prime flank was amplified in two steps: overlapping PCR products were created using Primers V90 V91 and V115 V116 (SEQ ID NOs:16, 17, 19 and 20, respectively) and lined via a unique SphI site present in both fragments. The flanking sequences were cloned into a single vector PVLCL (SEQ ID NO: 22).

To allow the Insertion of genes independent from those in the heavy chain locus analogous but hetero-specific frt based replacement system was designed. It contains in the 5′3′ direction a frt F3 site, the CMV EF1alpha hybrid promoter followed by the human alpha (1) antitrypsin gene, the hygromycin resistance marker, an wt frt site and an ATG deleted histidinol resistance marker. These elements were cloned into pVLCL to create pVLCLaathyg.

Since frt wt and F5 sites do not allow recombination, specific replacement vectors can exclusively target the heavy and light chain loci, providing a promoter and a start codon to the neomycin and histidinol resistance markers respectively. To increase selectivity, the replacement vectors contain start condons in different open reading frames relative to the frt site. As a result, incorporation of the vector into the wrong frt site does not result in resistance to the respective antibiotic.

PVLCLaathyg was transfected into PBG-04 using electroporation. Cells were seeded into a T75 flask and subjected to selection at 200 μg/ml Hygromycin. For 3 weeks. Resulting clones were isolated by dilution cloning and clones resulting from homologous exchange were identified by the absence of an immunoflourescence signal using a fluorescence-labelled anti human-lambda-chain antibody. The resulting cell clones are analyzed for the expression of alpha 1 antitrypsin. These clones are suitable for the coexpression of two independent transgenes which have to be expressed at high level. Preferably these genes are the heavy and light chain genes of an antibody. Within a single exchange reaction using flp recombinase, heavy and light chain genes can be directed to their respective locations.

1-17. (canceled)

18. A process for preparing a cell capable of stable high yield expression of a target gene product having an essentially human glycosylation pattern, which method comprises:
(a) selecting an immortalized human cell or human hybrid cell (starting cell) which is derived from B lymphocytes and is capable of stable high yield expression of an immunoglobulin (Ig) being non-essential to the starting cell;
(b) screening for the locus of the Ig gene within the genome of the starting cell;
(c) replacing the gene coding for the Ig with a first functional DNA sequence containing one or more recombinase recognition sites (RRS) to obtain a functionalized precursor cell; and
(d) integrating a second functional DNA sequence containing a DNA sequence coding for the target gene product into the functionalized precursor cell obtained in step (c) by use of a recombinase recognizing the RRSs incorporated with the first functional sequence, or
(e) directly replacing the gene coding for the Ig with a functional DNA sequence containing a DNA sequence coding for the target gene product.

19. The method of claim 18, wherein the starting cell secretes the Ig in an amount of at least 0.3 fmol/cell/d of a polypeptide chain.

20. The method of claim 19, wherein the starting cell secretes the Ig in an amount of more than 1 fmol/cell/d of a polypeptide chain.

21. The method of claim 18, wherein the starting cell is a human hybrid cell and the Ig gene is a human gene.

22. The method of claim 18 wherein the starting cell is selected from the group consisting of a human myeloma, a human hybridoma, and a human hetero-hybridoma cell.

23. The method of claim 22 where the starting cell is human-mouse hetero-hybridoma H-CB-P1 (DSM ACC 2104).

24. The method of claim 18, wherein the integration of the functional DNA sequence(s) is effected at a rearranged Ig locus of said starting cell.

25. The method of claim 24, where the integration of the functional DNA sequence(s) is effected at a rearranged immunoglobulin H locus of said starting cell.

26. The method of claim 24, where the integration of the functional DNA sequence(s) is effected at a λ locus of said starting cell.

27. The method of claim 18, wherein the locus of the Ig gene is a known locus.

28. The method of claim 18, wherein the locus of the Ig gene is determined by a screening procedure selected from the group consisting of microarray expression analysis, 2D protein gel electrophoresis, quantitative PCR, RNAse protection, northern blot, ELISA, western blot and combinations thereof.

29. The method of claim 27 wherein the locus of the Ig gene is selected as to provide for an essentially human glycosylation pattern.

30. The method of claim 18, wherein the replacement of the Ig gene is effected by an one step replacement strategy, wherein the starting cell is contacted with a vector construct containing the first functional sequence, said first functional sequence replacing the gene coding for the Ig.

31. The method of claim 18, wherein the replacement of the Ig gene is effected in a two- or multi-step strategy, wherein the gene coding for the Ig gene is deleted or inactivated and subsequently contacted with a vector construct containing the first functional sequence, said first functional sequence being incorporated at the site of the deleted/inactivated Ig.

32. The method of claim 18, wherein the first functional DNA sequence comprises one or more RRS(s) selected from the group consisting of loxP, frt, attL and attR sites of lambdoid phages, and recognition sites for resolvases or phage C31 integrase.

33. The method of claim 32 wherein the RRS(s) are capable of unidirectional integration

34. The method of claim 32 wherein the RRS(s) are selected from the group consisting of modified loxP sites and frt sites.

35. The method of claim 18, wherein the first functional DNA sequence further comprises functional sequences selected from the group consisting of marker sequences, secretion proteins, promoters, enhancers, splice signals, polyadenylation signals and IRES elements.

36. The method of claim 18, wherein the first functional DNA sequence is flanked in the vector by sequences selected for the group consisting of sequences that are homologous to the target gene or adjacent sequences.

37. The method of claim 18, wherein the integration of the second functional DNA sequence is effected by delivering a recombinases recognising the RRS(s) present in the first functional sequence together with, shortly before or after delivery of the second functional sequence.

38. The method of claim 18, wherein the integrase is selected from the group consistin of Cre, Flp, φC31 integrase and resolvase.

39. The method of claim 18, wherein the target gene product is selected from the group consisting of enzymes, hormones, cytokines, receptors, antibodies, antibody domains and fusion proteins comprising the gene product mentioned before.

40. The method of claim 18, wherein the second functional DNA sequence further comprises functional sequences selected from the group consisting of promoter sequences, marker sequences, splice donor and acceptor sequences and recombinase recognition sequences differing from the RRS of the first functional sequence.

41. The method of claim 18, wherein the gene coding for the Ig is directly replaced with a functional DNA sequence containing a DNA sequence coding for the target gene product.

42. A method for preparing a functionalized cell comprising the steps
(a) selecting an immortalized human cell or human hybrid cell (starting cell) which is derived from B lymphocytes and is capable of stable high yield expression of an immunoglobulin (Ig) being non-essential to the starting cell;
(b) screening for the locus of the Ig gene within the genome of the starting cell;
(c) replacing the gene coding for the Ig with a first functional DNA sequence containing one or more recombinase recognition sites (RRS) to obtain a functionalized precursor cell.

43. A functionalized cell as obtainable by the method of claim 25.

44. The functionalized cell of claim 43, which is derived from H-CB-P1 (DSM ACC2104).

45. A cell capable of high yield expression of a target gene product obtainable by the method of claim 18.

46. The cell of claim 45, which is derived from H-CB-P1 (DSM ACC2104).

47. The cell of claim 45, wherein the target gene product is an antibody.

48. The cell of claim 47, wherein the cell is PBG04 (DMS ACC2577).

49. The cell of claim 47, which is derived from H-CB-P1 (DSM ACC2104).

50. The cell of claim 47 further having its light chain inactivated or replaced with a gene coding for the same or a different target gene product.

51. A method for high yield expression of a target gene product which comprises cultivating a cell as defined in claim 45.

52. A target gene product obtained by cultivating a cell as defined in claim 42.

53. A target gene product obtained by cultivating a cell as defined in claim 45.
